(19) United States
(12) Patent Application Publication
(10) Pub. No.: US 2006/0100188 A1
(43) Pub. Date: May 11, 2006

(54) Treatment methods

(76) Inventors: Chen Zong, Metuchen, NJ (US); Benjamin Winograd, West Orange, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/268,160

(22) Filed: Nov. 7, 2005

Related U.S. Application Data
(60) Provisional application No. 60/626,258, filed on Nov. 9, 2004.

(51) Int. Cl. A61K 31/33

(52) U.S. Cl. 514/183

(57) ABSTRACT

There are disclosed methods for treating cancer in a patient in need of such treating comprising administering temozolomide according to improved dosing regimen and/or schedules based on the patient's MGMT level. Additional improved methods for treating patients with temozolomide are also disclosed.

This application claims priority from U.S. Provisional Application No. 60/626,258, filed Nov. 9, 2004, the entirety of which is incorporated by reference as if set forth fully herein.

### FIELD OF THE INVENTION

This invention describes novel methods and kits for treating subjects afflicted with a proliferative disease such as cancer, a tumor, or metastatic disease.

### BACKGROUND OF THE INVENTION

Discussion or citation of a reference herein shall not be construed as an admission that such reference is prior art to the present invention.

Stupp et al., J. Clin. Onc., 20(5):1375-1382 (2002), report that brain tumors comprise approximately 2% of all malignant diseases. However, it is stated that with an incidence of 5 per 100,000 persons, more than 17,000 cases are diagnosed every year in the United States, with approximately 13,000 associated deaths. In adults, Stupp et al. report, the most common histologies are grade 3 anaplastic astrocytoma and grade 4 glioblastoma multiforme (“GBM”). According to Stupp et al., the standard management of malignant gliomas involves cytoreduction through surgical resection, when feasible, followed by radiotherapy (RT) with or without adjuvant chemotherapy. However, Stupp et al. report that despite this multidisciplinary approach, the prognosis for patients with GBM remains poor. The median survival rates for GBM are reported to be typically in the range of 9 to 12 months, with 2-year survival rates in the range of only 8% to 12%.

Nitrosoureas are the main chemotherapeutic agents used in the treatment of malignant brain tumors. However, they have shown only modest antitumor activity. Although frequently prescribed in the United States, the benefit of adjuvant chemotherapy with single-agent carmustine (BCNU) or lomustine or the combination regimen procarbazine, lomustine, and vincristine has never been conclusively demonstrated.

Chemotherapeutic efficacy, the ability of chemotherapy to eradicate tumor cells without causing lethal host toxicity, depends on drug selectivity. One class of anticancer drugs, alkylating agents, cause cell death by binding to DNA which structurally distorts the DNA helical structure preventing DNA transcription and translation. In normal cells, the damaging action of alkylating agents can be repaired by cellular DNA repair enzymes, in particular O⁶-methylguanine-DNA methyltransferase (MGMT) also known as O⁶-alkylguanine-DNA-alkyltransferase (AGAT). The level of MGMT varies in tumor cells, even among tumors of the same type. The gene encoding MGMT is not commonly mutated or deleted. Rather, low levels of MGMT in tumor cells are due to an epigenetic modification; the MGMT promoter region is methylated, thus inhibiting transcription of the MGMT gene and preventing expression of MGMT.

Methylation has been shown by several lines of evidence to play a role in gene expression, cell differentiation, tumorigenesis, X-chromosome inactivation, genomic imprinting and other major biological processes. In eukaryotic cells, methylation of cytosine residues that are immediately 5′ to a guanosine, occurs predominantly in cytosine-guanine (CG) poor regions. In contrast, CpG islands remain unmethylated in normal cells, except during X-chromosome inactivation and parental specific imprinting where methylation of 5′ regulatory regions can lead to transcriptional repression. Expression of a tumor suppressor gene can also be abolished by de novo DNA methylation of a normally unmethylated CpG.

Hypermethylation of genes encoding DNA repair enzymes can serve as markers for predicting the clinical response to certain cancer treatments. Certain chemotherapeutic agents (including alkylating agents for example) inhibit cellular proliferation by cross-linking DNA, resulting in cell death. Treatment efforts with such agents can be thwarted and resistance to such agents develops because DNA repair enzymes remove the cross-linked structures. In view of the deleterious side effects of most chemotherapeutic drugs, and the ineffectiveness of certain drugs for various treatments, it is desirable to predict the clinical response to treatment with chemotherapeutic agents.

U.S. Pat. No. 6,773,897 discloses methods relating to chemotherapeutic treatment of a cell proliferative disorder. In particular, a method is provided for “predicting the clinical response to certain types of chemotherapeutic agents”, including specific alkylating agents. The method entails determination and comparison of the methylation state of nucleic acid encoding a DNA repair enzyme from a patient in need of treatment with that of a subject not in need of treatment. Any difference is deemed “predictive” of response. The method, however, offers no suggestion of how to improve clinical outcome for any patient with an unfavorable “prediction”.

Temozolomide is an alkylating agent available from Schering Corp. under the trade name of Temodar® in the United States and Temodal® in Europe. Temodar® Capsules for oral administration contain temozolomide, an imidazotetrazine derivative. The chemical name of temozolomide is 3,4-dihydro-3-methyl-4-oxoimidazo[5,1-d]-as-tetrazine-8-carboxamide (see U.S. Pat. No. 5,260,291). The cytotoxicity of temozolomide or metabolite of it, MTIC, is thought to be primarily due to alkylation of DNA. Alkylation (methylation) occurs mainly at the O⁶ and N⁷ positions of guanine.

Temodar® (temozolomide) Capsules are currently indicated in the United States for the treatment of adult patients with newly diagnosed gliobastoma multiforme as well as refractory anaplastic astrocytoma, i.e., patients at first relapse who have experienced disease progression on a drug regimen containing a nitrosourea and procarbazine. Temodal®) is currently approved in Europe for the treatment of patients with malignant glioma, such as glioblastoma multiforme or anaplastic astrocytoma showing recurrence or progression after standard therapy.

Although certain methods of treatment are effective for certain patients with proliferative diseases, there continues to be a great need for additional improved treatments, including in particular, targeted to particularly characterized patients. In view of the need for improved treatments for proliferative diseases, particularly cancers, novel methods of treatment would be a welcome contribution to the art. The present invention provides just such methods of treatment.

### SUMMARY OF THE INVENTION

One embodiment of the present invention provides a method for treating a patient having a proliferative disorder, comprising administering to the patient either a standard or enhanced dose intensity of temozolomide (TMZ) based upon the methylation state of the O⁶-methylguanine-DNA methyltransferase (MGMT) gene in a sample obtained from the patient. According to one mode of this embodiment of the invention, if the gene (e.g., the promoter region) encoding MGMT in a sample from the patient is methylated, a standard dose intensity of temozolomide is administered; however, if the gene encoding MGMT is not methylated (i.e., below the level of detection), an enhanced dose intensity of temozolomide is administered to the patient. One mode of this embodiment of the invention comprises: (1) assessing whether or not the MGMT gene in a sample from the patient is methylated and; (2) (a) if methylation of MGMT gene is detected, administering a standard dose intensity of temozolomide to the patient or (b) if methylation of MGMT gene is not detected, administering an enhanced dose intensity of temozolomide to the patient. Another mode of this embodiment of the invention comprises: administering an enhanced dose intensity to a patient in which methylation of the gene encoding MGMT is not detected.

As used herein the term “standard dose intensity” of temozolomide means a 5/28 dosing regimen, with a dosing schedule of 150-200 mg/m² of temozolomide per day, administered for 5 days in a 28 day cycle for a maximal total dose of 1000 mg/m²/4 weeks. This dosing regimen provides a “dose intensity” of 1.0.

As used herein the term “enhanced dose intensity” of temozolomide means a dosing regimen and/or dosing schedule which provides a dose intensity of temozolomide, which is 1.4-4.2, preferably 1.4-2.8, more preferably 1.8-2.8 times more intense (compared with the standard dose intensity). Non-limiting examples of dosing regimens and schedules which provide such enhanced dose intensities are illustrated in Table 1 and Table 2.

TABLE 1
TMZ Dosing Regimens and Dose Intensity

| | | | | Dose/ | |
|---|---|---|---|---|---|
| Regimen | | | Total Dose | wk | Dose |
| No. | Dosing Regimen | Dosing schedule | (mg/m²/4 wks) | (mg/m²) | Intensity |
| 1 | 5/28 | 150-200 mg/m², 5 | 1000 | 250 | 1 |
| | | days/28 day cycle | | | |
| | | (200 mg) | | | |
| 2 | High doses 250 mg/m² | 250 mg/m², 5/28, | 1250 | 312 | 1.2 |
| | for 5/28 | concomitant w/a | | | |
| | | growth factor | | | |
| 3 | 14/28 | 100 mg/m², 14 | 1400 | 350 | 1.4 |
| | | days/28 day cycle | | | |
| 4 | High doses 300 mg/m² | 300 mg/m², 5/28, | 1500 | 375 | 1.5 |
| | for 5/28 | concomitant w/a | | | |
| | | growth factor | | | |
| 5 | 21/28 | 75 mg/m², 21 | 1575 | 393.75 | 1.6 |
| | | days/28 day cycle | | | |
| 6 | 42/56 | 75 mg/m², 6 wks/8 wk | 3150 | 393.75 | 1.6 |
| | | cycle | | | |
| 7 | 21/28 | 85 mg/m², 21 | 1785 | 446.25 | 1.8 |
| | | days/28 day cycle | | | |
| 8 | High doses 350 mg/m² | 350 mg/m², 5/28, | 1750 | 437.5 | 1.8 |
| | for 5/28 | concomitant w/a | | | |
| | | growth factor | | | |
| 9 | 14 on/7 off | 100 mg/m², 14 | 1400* | 467 | 1.9 |
| | | days/21 day cycle | | | |
| 10 | High doses 400 mg/m² | 400 mg/m², 5/28, | 2000 | 500 | 2.0 |
| | for 5/28 | concomitant w/a | | | |
| | | growth factor | | | |
| 11 | 7/7 | 150 mg/m², 7 | 2100 | 525 | 2.1 |
| | | days/14 day cycle | | | |
| 12 | 21/28 | 100 mg/m², 21 | 2100 | 525 | 2.1 |
| | | days/28 day cycle | | | |
| 13 | 14/28 | 150 mg/m², 14 | 2100 | 525 | 2.1 |
| | | days/28 day cycle | | | |
| 14 | Continuous | 75 mg/m², daily | 2100 | 525 | 2.1 |
| | dosing | | | | |
| 15 | High doses 450 mg/m² | 450 mg/m², 5/28, | 2250 | 562.5 | 2.25 |
| | for 5/28 | concomitant w/a | | | |
| | | growth factor | | | |
| 16 | 14 on/7 off | 150 mg/m², 14 | 2100* | 700 | 2.8 |
| | | days/21 day cycle | | | |
| 17 | Continuous | 100 mg/m², daily | 2800 | 700 | 2.8 |
| | dosing | | | | |

*Represents total dose received in 3 week cycle

According to this embodiment of the present invention, when methylation of MGMT gene is not detected, a dosing regimen and/or dosing schedule which provide(s) a dose intensity of a least 1.6, or at least 1.8 times the standard dose intensity is preferred; under such condition, a dose intensity of at least 2.0 times the standard dose intensity is more preferred. In alternative embodiments, when methylation of the MGMT gene is not detected, dosing Regimen No. 9, No. 11, or No. 12 is preferred.

As would be understood by those skilled in the art, if the gene encoding MGMT is not methylated, the MGMT protein is expressed and can be detected (e.g., by Western blot, immunohistochemical techniques or enzymatic assays for MGMT activity, etc.) as detailed below herein or Northern blot for MGMT mRNA level (see for example, D'Atri et al., Journal of Pharmacological Exp. Ther., 294:664-671 (2000) or by RT-PCR for MGMT mRNA (see for example Patel et al., Mol. Cell Biol., 17(10):5813-5822 (1997); Watts et al., Mol. Cell. Biol., 17(9):5612-5619 (1997). Hence, according to an alternative embodiment of the invention, the presence or absence of the MGMT protein is assessed in a patient sample. A standard dose intensity or an enhanced dose intensity is administered to the patient based upon the absence or presence of the MGMT protein in the patient sample. In accord with this mode of the invention, if MGMT protein is detected, a dosing regimen and/or dosing schedule as shown in Table 1 which provides a dose intensity of at least 1.6, or at least 1.8 times the standard dose intensity, is preferred; under such condition, a dose intensity of at least 2.0 times the standard dose intensity is more preferred. In alternative embodiments, when MGMT protein is detected, dosing Regimen No. 9, No. 11, or No. 12 is preferred.

Another embodiment of the present invention provides a method for treating a patient having a proliferative disorder, comprising assigning the patient to and/or administering a dosing regimen of temozolomide to the patient based upon the degree or level of methylation of the MGMT gene in a sample obtained from the patient. According to one mode of this embodiment of the invention, the level of methylation of MGMT gene is assessed by determining the level of MGMT protein in a sample obtained from the patient. The level is classified as being “Low”, “Moderate”, or “High” and the patient is treated with one of the dosing regimens presented in Table 2 according to the Scheme set forth in Scheme 1 below.

TABLE 2
TMZ Dosing Regimen and Dose Intensity

| | | | | Dose/ | |
|---|---|---|---|---|---|
| Regimen | | | Total Dose | wk | Dose |
| No. | Dosing Regimen | Dosing schedule | (mg/m²/4 wks) | (mg/m²) | Intensity |
| 1 | 5/28 | 150-200 mg/m², 5 | 1000 | 250 | 1 |
| | | days/28 day cycle | | | |
| | | (200 mg) | | | |
| 2 | High doses 250 mg/m² | 250 mg/m², 5/28, | 1250 | 312 | 1.2 |
| | for 5/28 | concomitant w/a | | | |
| | | growth factor | | | |
| 3 | 14/28 | 100 mg/m², 14 | 1400 | 350 | 1.4 |
| | | days/28 day cycle | | | |
| 4 | High doses 300 mg/m² | 300 mg/m², 5/28, | 1500 | 375 | 1.5 |
| | for 5/28 | concomitant w/a | | | |
| | | growth factor | | | |
| 5 | 21/28 | 75 mg/m², 21 | 1575 | 393.75 | 1.6 |
| | | days/28 day cycle | | | |
| 6 | 42/56 | 75 mg/m², 6 wks/8 wk | 3150 | 393.75 | 1.6 |
| | | cycle | | | |
| 7 | 21/28 | 85 mg/m², 21 | 1785 | 446.25 | 1.8 |
| | | days/28 day cycle | | | |
| 8 | High doses 350 mg/m² | 350 mg/m², 5/28, | 1750 | 437.5 | 1.8 |
| | for 5/28 | concomitant w/a | | | |
| | | growth factor | | | |
| 9 | 14 on/7 off | 100 mg/m², 14 | 1400* | 467 | 1.9 |
| | | days/21 day cycle | | | |
| 10 | High doses 400 mg/m² | 400 mg/m², 5/28, | 2000 | 500 | 2.0 |
| | for 5/28 | concomitant w/a | | | |
| | | growth factor | | | |
| 11 | 7/7 | 150 mg/m², 7 | 2100 | 525 | 2.1 |
| | | days/14 day cycle | | | |
| 12 | 21/28 | 100 mg/m², 21 | 2100 | 525 | 2.1 |
| | | days/28 day cycle | | | |
| 13 | 14/28 | 150 mg/m², 14 | 2100 | 525 | 2.1 |
| | | days/28 day cycle | | | |
| 14 | Continuous | 75 mg/m², daily | 2100 | 525 | 2.1 |
| | dosing | | | | |
| 15 | High doses 450 mg/m² | 450 mg/m², 5/28, | 2250 | 562.5 | 2.25 |
| | for 5/28 | concomitant w/a | | | |
| | | growth factor | | | |
| 16 | 14 on/7 off | 150 mg/m², 14 | 2100* | 700 | 2.8 |
| | | days/21 day cycle | | | |
| 17 | Continuous | 100 mg/m², daily | 2800 | 700 | 2.8 |
| | dosing | | | | |
| 18 | High doses 250 mg/m² | 250 mg/m², for 7/7, | 3500 | 875 | 3.5 |
| | for 7/7 | concomitant with a | | | |
| | | growth factor | | | |
| 19 | High doses 300 mg/m² | 300 mg/m², for 7/7, | 4200 | 1050 | 4.2 |
| | for 7/7 | concomitant with a | | | |
| | | growth factor | | | |

*Represents total dose received in 3 week cycle

Scheme 1

| | Dose | Patient MGMT Protein Level | | |
|---|---|---|---|---|
| Regimen No. | Intensity | LOW | MODERATE | HIGH |
| 1 | 1 | + | | |
| 2 | 1.2 | + | | |
| 3 | 1.4 | | + | |
| 4 | 1.5 | | + | |
| 5 | 1.6 | | + | |
| 6 | 1.6 | | + | |
| 7 | 1.8 | | | + |
| 8 | 1.8 | | | + |
| 9 | 1.9 | | | + |
| 10 | 2.0 | | | + |
| 11 | 2.1 | | | + |
| 12 | 2.1 | | | + |
| 13 | 2.1 | | | + |
| 14 | 2.1 | | | + |
| 15 | 2.25 | | | + |
| 16 | 2.8 | | | + |
| 17 | 2.8 | | | + |
| 18 | 3.5 | | | + |
| 19 | 4.2 | | | + |

The degree or level of MGMT protein in a cell sample obtained from a patient can be assessed by any of a variety of methods. According to one mode of this embodiment of the invention, the level of MGMT protein expressed by cells of the patient is assessed by measurement of the MGMT protein, e.g., by Western blot using an antibody specific for MGMT. The level is compared to that expressed by normal lymphocytes known to express MGMT. Patient MGMT protein levels are classified as follows: Low=0-30% of the MGMT expressed by normal lymphocytes; Moderate=31-70% of the MGMT expressed by normal lymphocytes; and High=71-300% or higher of the MGMT expressed by normal lymphocytes. According to this embodiment, when the patient's MGMT protein level is High, Regimen No. 9, No. 11, or No. 12 is preferred.

According to another mode of this embodiment, the level of MGMT protein expressed by cells of the patient is assessed by measurement of the MGMT protein using an immunohistochemistry technique on a defined number of patient cells, e.g., employing a labeled antibody specific for MGMT and comparing the level with that expressed by the same defined number of normal lymphocytes known to express MGMT. Patient MGMT levels are classified as follows: Low=0-30% of the MGMT expressed by normal lymphocytes; Moderate=31-70% of the MGMT expressed by normal lymphocytes; and High=71-300% or higher of the MGMT expressed by normal lymphocytes. According to this embodiment, when the patient's MGMT protein level is High, Regimen No. 9, No. 11, or No. 12 is preferred.

According to yet another mode of this embodiment, the level of MGMT is assessed by enzymatic assay of the MGMT expressed by cells in a patient sample. For example, protein is immunoprecipitated from lysate of cells in a patient sample and the enzymatic activity, i.e., the ability to methylate the O⁶ or N⁷ guanine position of DNA is assessed and compared to that of normal lymphocytes known to express MGMT. Patient MGMT levels are classified as follows: Low=0-30% of the MGMT enzymatic activity of normal lymphocytes; Moderate=31-70% of the MGMT enzymatic activity of normal lymphocytes; and High=71-300% or higher of the MGMT enzymatic activity of normal lymphocytes. According to this embodiment, when the patient's MGMT enzymatic activity level is High, Regimen No. 9, No. 11, or No. 12 is preferred.

In an alternative embodiment, the specific activity of MGMT is assessed and based on a comparison with cell lines known to express MGMT classified as follows: Low=less than 20 fmol/mg; Moderate=20-60 fmol/mg; or High=greater than 60 fmol/mg; where the specific activity of MGMT in LOX cells is 6-9 fmol/mg, in DAOY cells is 60-100 fmol/mg, and in A375 cells is 80-150 fmol/mg. According to this alternative embodiment, when the patient's MGMT enzymatic activity level is High, Regimen No. 9, No. 11, or No. 12 is preferred.

According to yet another mode of this embodiment, the level of methylation of MGMT is assessed by quantitative determination of the methylation of the gene encoding MGMT. The quantitative technique called Combined Bisulfite Restriction Analysis (COBRA) (Xiong et al., Nuc. Acids Res., 25:2532-2534 (1997)) is useful for this mode. The level of methylation of gene encoding MGMT in cells of the patient is compared to that of an equivalent number of cells of normal lymphocytes known to express MGMT. As would be understood by those skilled in the art, normal lymphocytes expressing MGMT have a low level of methylation of the MGMT gene; conversely, cells with high levels of methylation of the MGMT gene express low levels of the MGMT protein (see for example, Costello et al., J. Biol. Chem., 269(25):17228-17237 (1994); Qian et al., Carcinogen, 16(6):1385-1390 (1995)). Patient methylated MGMT gene levels are classified as follows: Low=0-20% of the CpGs in the promoter region of the MGMT gene are methylated; Moderate=21-50% of the CpGs in the promoter region of the MGMT gene are methylated; and High=51-100% of the CpGs in the promoter region of the MGMT gene are methylated. Once the level of methylation of MGMT gene is assessed and patients are classified, patients are treated using a dosing regimen set forth in Table 2 using the Scheme set forth in Scheme 2.

Scheme 2

| | Dose | Patient MGMT Gene Methylation Level | | |
|---|---|---|---|---|
| Regimen No. | Intensity | LOW | MODERATE | HIGH |
| 1 | 1 | | | + |
| 2 | 1.2 | | | + |
| 3 | 1.4 | | + | |
| 4 | 1.5 | | + | |
| 5 | 1.6 | | + | |
| 6 | 1.6 | | + | |
| 7 | 1.8 | + | | |
| 8 | 1.8 | + | | |
| 9 | 1.9 | + | | |
| 10 | 2.0 | + | | |
| 11 | 2.1 | + | | |
| 12 | 2.1 | + | | |
| 13 | 2.1 | + | | |
| 14 | 2.1 | + | | |
| 15 | 2.25 | + | | |
| 16 | 2.8 | + | | |
| 17 | 2.8 | + | | |
| 18 | 3.5 | + | | |
| 19 | 4.2 | + | | |

According to this mode of this embodiment, when the patient's level of methylation of the MGMT gene is Low, Regimen No. 9, No. 10, or No. 11 is preferred.

According to yet another mode of this embodiment, the level of methylation of the MGMT gene is assessed quantitatively to determine what percentage of the MGMT allele in a sample is methylated. See for e.g., U.S. Pat. No. 6,331,393, issued Dec. 18, 2001; Eads et al., Nuc. Acids Res., 28(8):e32 (2000), incorporated herein by reference, for illustrative quantitative methods useful for this mode of the invention.

Patient MGMT methylation levels are determined and classified as follows: Low=0-20% of the cells have methylated MGMT gene; Moderate=21-50% of the cells have methylated MGMT gene; and High=51-100% of the cells have methylated MGMT gene. Once the level of methylation is assessed and patients are classified, patients are treated using a dosing regimen set forth in Table 2 using the Scheme set for in Scheme 2 supra.

Another alternative embodiment of the present invention provides an improved method for treating a patient having a proliferative disorder, comprising administering to the patient a dose intensity of temozolomide of 1.4-2.8 compared to the standard dose intensity according to Regimens 3-16 of Table 1 supra.

As used herein, “treating” or “treatment” is intended to mean mitigating or alleviating a cell proliferative disorder in a mammal such as a human.

A cell proliferative disorder as described herein may be a neoplasm. Such neoplasms are either benign or malignant. The term “neoplasm” refers to a new, abnormal growth of cells or a growth of abnormal cells that reproduce faster than normal. A neoplasm creates an unstructured mass (a tumor) which can be either benign or malignant. The term “benign” refers to a tumor that is noncancerous, e.g., its cells do not invade surrounding tissues or metastasize to distant sites. The term “malignant” refers to a tumor that is cancerous, metastastic, invades contiguous tissue or is no longer under normal cellular growth control. In preferred embodiments, the methods and kits of the invention are used to treat proliferative disorders including but not limited to melanoma, glioma, prostate, lung cancer, breast cancer, ovarian, testicular cancer, liver, kidney, spleen, bladder, colorectal and/or colon cancer, head and neck, carcinoma, sarcoma, lymphoma, leukemia or mycosis fungoides. In more preferred embodiments, the methods and kits of the invention are used to treat melanoma, glioma, lung cancer, lymphoma, colorectal and/or colon cancer, head and neck or ovarian cancer.

As used herein, a “sample” obtained from a patient can be obtained as or isolated from tumor tissue, brain tissue, cerebrospinal fluid, blood, plasma, serum, lymph, lymph nodes, spleen, liver, bone marrow, or any other biological specimen containing either MGMT protein or nucleic acid of the MGMT gene.

The present invention also provides kits for treating patients with proliferative disorders. The kits comprise: (1) reagents used in the methods of the invention; and (2) instructions to carry out the methods as described herein. The kits can further comprise temozolomide.

As would be understood by those skilled in the art, the novel methods and kits of the present invention for treating patients with proliferative disorders using temozolomide can be used as monotherapy or can be used in combination with radiotherapy and/or other cytotoxic and/or cytostatic agent(s) or hormonal agent(s) and/or other adjuvant therapy(ies).

### BRIEF DESCRIPTION OF THE DRAWINGS

FIG. 1 illustrates the number of DAOY human glioma cell colonies, a high MGMT level cell line, present after a 4-day cycle of TMZ treatment, where TMZ was administered according to one of two different dosing schedules: (i) continuous daily dosing (Day 1-4); or (ii) single pulse dosing (Day 1).

FIG. 2 illustrates the number of A375 human melanoma cell colonies, a high MGMT level cell line, present after a 4-day cycle of TMZ treatment, where TMZ was administered according to one of two different dosing schedules: (i) continuous daily dosing (Day 1-4); or (ii) single pulse dosing (Day 1).

FIG. 3A illustrates the number of LOX human melanoma cell colonies, a low MGMT level cell line, present after a 4-day cycle of TMZ treatment, where TMZ was administered according to one of two different dosing schedules: (i) continuous daily dosing (Day 1-4); or (ii) single pulse dosing (Day 1).

FIG. 3B illustrates the number of LOX human melanoma cell colonies, a low MGMT level cell line, present after an 8-day cycle of TMZ treatment, where TMZ was administered according to one of three different dosing schedules: (i) continuous daily dosing (Day 1-8); (ii) dosing for 2 consecutive days (Day 1-2); or (ii) intermittent dosing for 2 days (Day 1, Day 5).

FIG. 4A illustrates the level of MGMT enzymatic activity in A375 human melanoma cells, a high MGMT level cell line, following TMZ treatment.

FIG. 4B illustrates the level of MGMT protein in A375 human melanoma cells, a high MGMT level cell line, following TMZ treatment. Lanes 1-4 reflect cell lysates prepared after 72 hours of TMZ treatment. Lanes 5-8 reflect cell lysates prepared after 72 hours of TMZ treatment followed by an additional 72 hours without TMZ treatment.

FIG. 5A illustrates the mean tumor growth curves of DAOY human glioma xenograft tumors, a high MGMT level cell line, following TMZ treatment for two consecutive 15-day cycles of continuous daily dosing (Day 1-15 (first cycle), Day 16-30 (second cycle)); where the total dose of TMZ administered was 0, 360, 540, or 810 mg per kg (mpk).

FIG. 5B illustrates the mean tumor growth curves of DAOY human glioma xenograft tumors, a high MGMT level cell line, following TMZ treatment for two consecutive 15-day cycles of dosing for 5 consecutive days (Day 1-5 (first cycle); Day 16-20 (second cycle)); where the total dose of TMZ administered was 0, 360, 540, or 810 mpk.

FIG. 5C illustrates mean tumor growth curves of DAOY human glioma xenograft tumors, a high MGMT level cell line, following TMZ treatment for two consecutive 15-day cycles of intermittent dosing for 5 days (Day 1, 4, 7, 10, 13 (first cycle); Day 16, 19, 22, 25, 28 (second cycle)); where the total dose of TMZ administered was 0, 360, 540, or 810 mpk.

FIG. 6 illustrates the individual tumor volume of A375 human melanoma xenograft tumors, a high MGMT level cell line, on Day 15 following a 15-day cycle of TMZ treatment, where TMZ was administered according to one of three different dosing schedules: (i) continuous daily dosing (Day 1-15); (ii) dosing for 5 consecutive days (Day 1-5); or (ii) intermittent dosing for 5 days (Day 1, 4, 7, 10, 13); where the total dose of TMZ administered was 0, 180, 270, or 405 mpk.

FIG. 7 illustrates the individual tumor volume of LOX human melanoma xenograft tumors, a low MGMT level cell line, on Day 18 following a 12-day cycle of TMZ treatment, where TMZ was administered according to one of two different dosing schedules: (i) continuous daily dosing (Day 1-12); or (ii) dosing for 4 consecutive days (Day 1-4); where the total dose of TMZ administered was 0, 36, 72, or 144 mpk.

FIG. 8 illustrates the level of MGMT enzymatic activity in individual DAOY human glioma xenograft tumors, a high MGMT level cell line, following TMZ treatment for 5 consecutive days (where the total dose of TMZ administered was 0 or 405 mpk); as well as the level of MGMT enzymatic activity in untreated DAOY human glioma cells harvested from cell culture. C1, C2, and C3 represent tumors isolated from three different mice that had been treated with vehicle, while T1, T2, T3 represent tumors isolated from another three different mice that had been treated with TMZ.

FIG. 9 illustrates the number of DAOY human glioma cell colonies, a high MGMT level cell line, present after a 4-day cycle of TMZ treatment.

FIG. 10 illustrates the number of A375 human melanoma cell colonies, a high MGMT level cell line, present after a 4-day cycle of TMZ treatment.

FIG. 11 illustrates the number of LOX human melanoma cell colonies, a low MGMT level cell line, present after a 4-day cycle of TMZ treatment.

FIG. 12 illustrates the number of LOX human melanoma cell colonies, a low MGMT level cell line, present after an 8-day cycle of TMZ treatment.

FIG. 13 illustrates the % inhibition of DAOY human glioma cell colony formation after a 4-day cycle of TMZ treatment.

FIG. 14 illustrates the % inhibition of A375 human melanoma cell colony formation after a 4-day cycle of TMZ treatment.

FIG. 15 illustrates the % inhibition of LOX human melanoma cell colony formation after a 4-day cycle of TMZ treatment.

FIG. 16 illustrates the % inhibition of LOX human melanoma cell colony formation after an 8-day cycle of TMZ treatment.

FIG. 17 illustrates the tumor volume in DAOY human glioma xenograft tumors, a high MGMT level cell line, following TMZ treatment over two 15-day cycles where the dose intensity was 1, 1.5, or 2.25.

FIG. 18 illustrates the tumor volume in A375 human melanoma xenograft tumors, a high MGMT level cell line, following TMZ treatment over a 15-day cycle where the dose intensity was 1, 1.5, or 2.25.

FIG. 19 illustrates the tumor volume in LOX human melanoma xenograft tumors, a low MGMT level cell line, following TMZ treatment over a 12-day cycle where the dose intensity was 1, 2, or 4.

FIG. 20 illustrates the tumor growth inhibition (%) of DAOY human glioma xenograft tumors following TMZ treatment over two 15-day cycles where the dose intensity was 1, 1.5, or 2.25.

FIG. 21 illustrates the tumor growth inhibition (%) of A375 human melanoma xenograft tumors following TMZ treatment over a 15-day cycle where the dose intensity was 1, 1.5, or 2.25.

FIG. 22 illustrates the tumor growth inhibition (%) of DAOY human melanoma xenograft tumors following TMZ treatment over a 12-day cycle where the dose intensity was 1, 2, or 4.

### DETAILED DESCRIPTION OF THE INVENTION

The present invention provides novel methods and kits for treating a patient with a proliferative disorder, comprising administering to the patient a standard or a more intense dose intensity based upon the methylation state of the MGMT gene in a sample obtained from the patient. In certain embodiments, the methylation state is assessed by a determination of whether or not the MGMT gene is methylated. In certain other embodiments, the methylation state is assessed by a quantitative determination of the level of methylation of the MGMT gene. In yet other embodiments, the methylation state is assessed by determination of whether or not MGMT protein is expressed or determination of the level of MGMT protein expressed or by measurement of the enzymatic activity of MGMT in the patient sample.

One embodiment of the present invention provides a method for treating a patient having a proliferative disorder, comprising administering to the patient either a standard or enhanced dose intensity of temozolomide (TMZ) based upon the methylation state of the O⁶-methylguanine-DNA methyltransferase (MGMT) gene in a sample obtained from the patient. According to one mode of this embodiment of the invention, if the gene (e.g., the promoter region) encoding MGMT in a sample from the patient is methylated, a standard dose intensity of temozolomide is administered; however, if the gene encoding MGMT is not methylated (i.e., below the level of detection), an enhanced dose intensity of temozolomide is administered to the patient. One mode of this embodiment of the invention comprises: (1) assessing whether or not the MGMT gene in a sample from the patient is methylated and; (2) (a) if methylation of MGMT gene is detected, administering a standard dose intensity of temozolomide to the patient or (b) if methylation of MGMT gene is not detected, administering an enhanced dose intensity of temozolomide to the patient. Another mode of this embodiment of the invention comprises: administering an enhanced dose intensity to a patient in which methylation of the gene encoding MGMT is not detected.

As used herein the term “standard dose intensity” of temozolomide means a 5/28 dosing regimen, with a dosing schedule of 150-200 mg/m² of temozolomide per day, administered for 5 days in a 28 day cycle for a maximal total dose of 1000 mg/m²/4 weeks. This dosing regimen provides a “dose intensity” of 1.0.

As used herein the term “enhanced dose intensity” of temozolomide means a dosing regimen and/or dosing schedule which provides a dose intensity of temozolomide, which is 1.2-2.8 times more intense (compared with the standard dose intensity). Non-limiting examples of dosing regimens and schedules which provide such enhanced dose intensities are illustrated in Table 1 and Table 2.

According to this embodiment of the present invention, when methylation of MGMT gene is not detected, a dosing regimen and/or dosing schedule which provide(s) a dose intensity of a least 1.6, or at least 1.8 times the standard dose intensity is preferred; under such condition, a dose intensity of at least 2.0 times the standard dose intensity is more preferred. In alternative embodiments, when methylation of the MGMT gene is not detected, dosing Regimen No. 9, No. 11, or No. 12 is preferred.

Assessing whether or not the MGMT gene is methylated can be performed using any method known to one skilled in the art. Techniques useful for detecting methylation of a gene or nucleic acid include, but are not limited to those described by Ahrendt et al., J. Natl. Cancer Inst., 91:332-339 (1999); Belsinky et al., Proc. Natl. Acad. Sci. U.S.A., 95:11891-11896 (1998), Clark et al., Nucleic Acids Res., 22:2990-2997 (1994); Herman et al., Proc Natl Acad Sci U.S.A., 93:9821-9826 (1996); Xiong and Laird, Nucleic Acids Res., 25:2532-2534 (1997); Eads et al., Nuc. Acids. Res., 28:e32 (2002); Cottrell et al., Nucleic Acids Res., 32:1-8 (2004). All references cited herein are incorporated herein by reference.

Methylation-specific PCR (MSP; Herman et al., Proc. Natl. Acad Sci. USA, 93(18):9821-9826 (1996); Esteller et al., Cancer Res., 59:793-797 (1999)) see also U.S. Pat. No. 5,786,146, issued Jul. 28, 1998; U.S. Pat. No. 6,017,704, issued Jan. 25, 2000; U.S. Pat. No. 6,200,756, issued Mar. 13, 2001; and U.S. Pat. No. 6,265,171, issued Jul. 24, 2001; U.S. Pat. No. 6,773,897 issued Aug. 10, 2004; the entire contents of each of which is incorporated herein by reference can rapidly assess the methylation status of virtually any group of CpG sites within a CpG island, independent of the use of methylation-sensitive restriction enzymes. This assay entails initial modification of DNA by sodium bisulfite, converting all unmethylated, but not methylated, cytosines to uracil, and subsequent amplification with primers specific for methylated versus unmethylated DNA. MSP requires only small quantities of DNA, is sensitive to 0.1% methylated alleles of a given CpG island locus, and can be performed on DNA extracted from paraffin-embedded samples. MSP eliminates the false positive results inherent to previous PCR-based approaches which relied on differential restriction enzyme cleavage to distinguish methylated from unmethylated DNA. This method is very simple and can be used on small amounts of tissue or a few cells. As would be understood by those skilled in the art, if the gene encoding MGMT is not methylated, the MGMT protein is expressed and can be detected (e.g., by Western blot, immuno-histochemical techniques or enzymatic assays for MGMT activity, etc.) as detailed below herein. Hence, according to an alternative embodiment of the invention, the presence or absence of the MGMT protein is assessed in a patient sample. A standard dose intensity or an enhanced dose intensity is administered to the patient based upon the absence or presence of the MGMT protein in the patient sample. In accord with the absence mode of the invention, if MGMT protein is detected, a dosing regimen and/or dosing schedule as shown in Table 1 which provides a dose intensity of at least 1.6, or at least 1.8 times the standard dose intensity, is preferred; under such condition, a dose intensity of at least 2.0 times the standard dose intensity is more preferred. In alternative embodiments, when MGMT protein is detected, dosing Regimen No. 9, No. 11, or No. 12 is preferred.

An illustrative example of a Western blot assay useful for this embodiment of the invention to measure the level of MGMT protein in patient samples is presented in U.S. Pat. No. 5,817,514 by Li et al., the entire disclosure of which is incorporated herein by reference. Li et al. described monoclonal antibodies able to specifically bind either to native human MGMT protein or to human MGMT protein having an active site which is alkylated. An illustrative example of an immunohistochemical technique useful for this embodiment of the invention to measure the level of MGMT protein in patient samples is presented in U.S. Pat. No. 5,407,804, the entire disclosure of which is incorporated herein by reference. Monoclonal antibodies are disclosed which are able to specifically bind to the MGMT protein in single cell preparations (immunohistochemical staining assays) and in cell-extracts (immunoassays). The use of fluorescent read out coupled with digitization of the cell image is described and allows for quantitative measurement of MGMT levels in patient and control samples, including but not limited to tumor biopsy samples.

Useful techniques for measuring the enzymatic acitivity of MGMT protein include but are not limited to methods described by: Myrnes et al., Carcinogenesis, 5:1061-1064 (1984); Futscher et al., Cancer Comm., 1: 65-73 (1989); Kreklaw et al., J. Pharmacol. Exper. Ther., 297(2):524-530 (2001); and Nagel et al., Anal. Biochem., 321(1):38-43 (2003), the entire disclosures of which are incorporated herein in their entireties.

Another embodiment of the present invention provides a method for treating a patient having a proliferative disorder, comprising assigning the patient to and/or administering a dosing regimen of temozolomide to the patient based upon the degree or level of methylation of the MGMT gene in a sample obtained from the patient. According to one mode of this embodiment of the invention, the level of methylation of MGMT gene is assessed by determining the level of MGMT protein in a sample obtained from the patient. The level is classified as being “Low”, “Moderate”, or “High” and the patient is treated with one of the dosing regimens presented in Table 2 according to the Scheme set forth in Scheme 1 supra.

The degree or level of MGMT protein in a cell sample obtained from a patient can be assessed by any of a variety of methods (see supra herein).

According to one mode of this embodiment of the invention, the level of MGMT protein expressed by cells of the patient is assessed by measurement of the MGMT protein, e.g., by Western blot using an antibody specific to MGMT, see for example, U.S. Pat. No. 5,817,514 (supra) by Li et al. for a description of a Western blot assay to determine MGMT level. The level is compared to that expressed by normal lymphocytes known to express MGMT. Patient MGMT protein levels are classified as follows: Low=0-30% of the MGMT expressed by normal lymphocytes; Moderate=31-70% of the MGMT expressed by normal lymphocytes; and High=71-300% or higher of the MGMT expressed by normal lymphocytes. According to this embodiment, when the patient's MGMT protein level is High, Regimen No. 9, No. 11, or No. 12 is preferred.

According to another mode of this embodiment, the level of MGMT protein expressed by cells of the patient is assessed by measurement of the MGMT protein using an immunohistochemistry technique on a defined number of patient cells, e.g., employing a labeled antibody specific for MGMT and compairing the level with that expressed by the same defined number of normal lymphocytes known to express MGMT (see, for example, U.S. Pat. No. 5,407,804 by Yarosh for a description of useful quantitative immunohistochemical assays. Patient MGMT levels are classified as follows: Low=0-30% of the MGMT expressed by normal lymphocytes; Moderate=31-70% of the MGMT expressed by normal lymphocytes; and High=71-300% or higher of the MGMT expressed by normal lymphocytes. According to this embodiment, when the patient's MGMT protein level is High, Regimen No. 9, No. 11, or No. 12 is preferred.

According to yet another mode of this embodiment, the level of MGMT is assessed by enzymatic assay of the MGMT expressed by cells in a patient sample. For example, protein is immunoprecipitated from lysate of cells in a patient sample and the enzymatic activity, i.e., the ability to methylate the O⁶ or N⁷ guanine position of DNA is assessed and compared to that of normal lymphocytes known to express MGMT (see supra for description of useful assays to determine enzymatic activity of MGMT protein). Patient MGMT levels are classified as follows: Low=0-30% of the MGMT enzymatic activity of normal lymphocytes; Moderate=31-70% of the MGMT enzymatic activity of normal lymphocytes; and High=71-300% or higher of the MGMT enzymatic activity of normal lymphocytes. According to this embodiment, when the patient's MGMT protein level is High, Regimen No. 9, No. 11, or No. 12 is preferred.

In an alternative embodiment, the specific activity of MGMT is assessed and based on a comparison with cell lines known to express MGMT classified as follows: Low=less than 20 fmol/mg; Moderate=20-60 fmol/mg; or High=greater than 60 fmol/mg; where the specific activity of MGMT in LOX cells is 6-9 fmol/mg, in DAOY cells is 60-100 fmol/mg, and in A375 cells is 80-150 fmol/mg. According to this alternative embodiment, when the patient's MGMT enzymatic activity level is High, Regimen No. 9, No. 11, or No. 12 is preferred.

According to yet another mode of this embodiment, the level of methylation of MGMT is assessed by quantitative determination of the methylation of the gene encoding MGMT. The quantitative technique called COBRA (Xiong et al., Nuc. Acids Res., 25:2532-2534 (1997)) is useful for this mode. The “methyl light” technique of Eads et al., Nuc. Acids Res., 28(8):e32 (2000); U.S. Pat. No. 6,331,393 is also useful for quantitative determination for this mode. The level of methylation of gene encoding MGMT in cells of the patient is compared to that of an equivalent number of cells of normal lymphocytes known to express MGMT. As would be understood by those skilled in the art, normal lymphocytes expressing MGMT have a low level of methylation of the MGMT gene; conversely, cells with high levels of methylation of the MGMT gene express low levels of the MGMT protein (see for example, Costello et al., J. Biol. Chem., 269(25):17228-17237 (1994); Qian et al., Carcinogen, 16(6):1385-1390 (1995)). Patient methylated MGMT gene levels are classified as follows: Low=0-20% of the CpGs in the promoter region of the MGMT gene are methylated; Moderate=21-50% of the CpGs in the promoter region of the MGMT gene are methylated; and High=51-100% of the CpGs in the promoter region of the MGMT gene are methylated. Once the level of methylation of MGMT gene is assessed and patients are classified, patients are treated using a dosing regimen set forth in Table 2 supra using the Scheme set forth in Scheme 2 supra.

According to this mode of this embodiment, when the patient's level of methylation of the MGMT gene is Low, Regimen No. 9, No. 10, or No. 11 is preferred.

As indicated above, the quantitative technique called COBRA (Xiong et al., Nucleic Acids Res., 25(12):2532-2534 (1997)) can be used to determine quantitatively DNA methylation levels at specific gene loci in small amounts of genomic DNA. Restriction enzyme digestion is used to reveal methylation-dependent sequence differences in PCR products of sodium bisulfite-treated DNA. (Tano et al., Proc. Natl. Acad. Sci. USA, 87:686-690 (1990) describes isolation and sequence of the human MGMT gene). Methylation levels in original DNA sample are represented by the relative amounts of digested and undigested PCR product in a linearly quantitative fashion across a wide spectrum of DNA methylation levels. This technique can be reliably applied to DNA obtained from microdissected paraffin-embedded tissue samples. COBRA thus combines the powerful features of ease of use, quantitative accuracy, and compatibility with paraffin sections.

An illustrative example of a RT-PCR assay useful for assessing the level of MGMT mRNA is described in Watts et al., Mol. Cell. Biol., 17(9):5612-5619 (1997). In brief, total cellular RNA is isolated by guanidium isothiocyanate cell lysis followed by centrifugation through a 5.7 M CsCl gradient for 2.5 hr at 205,000×g. RNA is quantitated in a Beckman TL-100 spectrophotometer by measurements of absorbance at 260 nm. Total cellular RNA is reverse transcribed by incubating a 40 μl reaction mixture composed of 200 ng of RNA; 1× PCR buffer (10 mM Tris [pH 8.3], 50 mM KCl, 1.5 mM MgCl₂); 1 mM each dATP, dCTP, dGTP, and dTTP; 200 pmol of random hexamer, 40 U of RNasin, and 24 U of avian myeloblastosis virus reverse transcriptase (Boehringer Mannheim, Indianapolis, Ind.) at 42° C. for 60 min. The reaction is then stopped by incubation at 99° C. for 10 min. MGMT-specific PCR is performed by adding 80 μl of amplification reaction buffer (1× PCR buffer, 25 pmol of MGMT-specific primers and/or a control sequence, and 2 U of Taq DNA polymerase) to 20 μl of the reverse transcription reaction mixture followed by incubation at 94° C. for 5 min; 30 cycles of 94° C. for 1 min, 60° C. for 15 s, and 72° C. for 1 min; a final extension at 72° C. for 5 min; and a quick chill to 4° C. For example, the upstream primer sequence from exon 4 (nt 665 to 684) of the MGMT gene can be used. Nucleotide positions can be derived from the cDNA sequence (Tano et al., Proc. Natl. Acad. Sci. USA, 87:686-690 (1990)). A control primer sequence can be employed in the same cDNA reaction (e.g., primers for the histone 3.3 gene). For analysis, 10% of the respective PCR products are separated through a 3% agarose gel and visualized by ethidium bromide staining.

An illustrative example of a Northern blot useful for assessing the level of MGMT mRNA is described in D'Atri et al., Journal of Pharmacological Exp. Ther., 294:664-671 (2000). In brief, total cellular RNA is extracted using the guanidine thiocyanate-phenol-chloroform method (Chomczynski and Sacchi, Anal Biochem, 162(1):156-159 (1987)). Subsequently, an aliquot is fractionated by electrophoresis on a formaldehyde-containing 1.2% agarose gel and RNA integrity confirmed by visualization following ethidium bromide staining of the gel. RNA is then transferred to a nylon membrane (Genescreen Plus; New England Nuclear, Boston, Mass.) and hybridized at 42° C. for 24 h with a ³²P-labeled MGMT probe and a control probe (e.g., glyceraldehyde-3 phosphate dehydrogenase (GAPDH)). For example, the MGMT probe may be a polymerase chain reaction-derived cDNA probe obtained after reverse transcription of the RNA from Molt-4 cells (Lacal et al., J Pharmacol Exp Ther, 279(1):416-422 (1996)). After washing with 0.1× standard saline citrate (10 mM sodium chloride, 1.5 mM sodium citrate) at room temperature for 30 min, the blotted membranes are exposed to x-ray films (Kodak, Rochester, N.Y.) at −80° C. Bidimensional densitometry of the blots may be performed using an Imaging densitometer GS-670 (Bio-Rad, Richmond, Calif.).

Another alternative embodiment of the present invention provides an improved method for treating a patient having a proliferative disorder, comprising administering to the patient a dose intensity of temozolomide of 1.4-2.8 compared to the standard dose intensity according to Regimens 3-16 of Table 1 supra.

Also encompassed within the scope of the present invention are methods of administering temozolomide as described above herein in combination with a PARP inhibitor. The compelling evidence for the role of poly(ADP-ribose) polymerase(s) (PARP) in the cellular reaction to genotoxic stress was the stimulus to develop inhibitors as therapeutic agents to potentiate DNA-damaging anticancer therapies. Over the last two decades potent PARP inhibitors have been developed using structure activity relationships (SAR) and crystal structure analysis. These approaches have identified key desirable features for potent inhibitor-enzyme interactions. The resulting PARP inhibitors are up to 1,000 times more potent than the classical benzamides. These novel potent inhibitors have helped define the therapeutic potential of PARP inhibition. PARP inhibitors increase the antitumour activity of three classes of anticancer agents including temozolomide. A PARP inhibitor can be administered either prior to, concomitantly with or after administration of temozolomide as described herein. Exemplary PARP inhibitors include CEP-6800 (Cephalon; described in Miknyoczki et al., Mol Cancer Ther, 2(4):371-382 (2003)); 3-aminobenzamide (also known as 3-AB; Inotek; described in Liaudet et al., Br J Pharmacol, 133(8):1424-1430 (2001)); PJ34 (Inotek; described in Abdelkarim et al., Int J Mol Med, 7(3):255-260 (2001)); 5-iodo-6-amino-1,2-benzopyrone (also known as INH(2)BP; Inotek; described in Mabley et al., Br J Pharmacol, 133(6):909-919 (2001), GPI 15427 (described in Tentori et al., Int J Oncol, 26(2):415-422 (2005)); 1,5-dihydroxyisoquinoline (also known as DIQ; described in Walisser and Thies, Exp Cell Res, 251(2):401-413 (1999); 5-aminoisoquinolinone (also known as 5-AIQ; described in Di Paola et al., Eur J Pharmacol, 492(2-3):203-210 (2004); AG14361 (described in Bryant and Helleday, Biochem Soc Trans, 32(Pt 6):959-961 (2004); Veuger et al., Cancer Res, 63(18):6008-6015 (2003); and Veuger et al., Oncogene, 23(44):7322-7329 (2004)); ABT-472 (Abbott); INO-1001 (Inotek); AAI-028 (Novartis); KU-59436 (KuDOS; described in Farmer et al., “Targeting the DNA repair defect in BRCA mutant cells as a therapeutic strategy,” Nature, 434(7035):917-921 (2005)); and those described in Jagtap et al., Crit Care Med, 30(5):1071-1082 (2002); Loh et al., Bioorg Med Chem Lett, 15(9):2235-2238 (2005); Ferraris et al., J Med Chem, 46(14):3138-3151 (2003); Ferraris et al., Bioorg Med Chem Lett, 13(15):2513-2518 (2003); Ferraris et al., Bioorg Med Chem, 11(17):3695-3707 (2003); Li and Zhang IDrugs, 4(7):804-812 (2001); Steinhagen et al., Bioorg Med Chem Lett, 12(21):3187-3190 (2002)); WO 02/06284 (Novartis); and WO 02/06247 (Bayer). In addition, a high-throughput screen for PARP-1 inhibitors is described in Dillon et al., J Biomol Screen, 8(3):347-352 (2003).

One treatment method involves improving the effectiveness of temozolomide and/or radiotherapy administered to a mammal in the course of therapeutic treatment, comprising administering to the mammal an effective amount of a PARP-inhibiting agent (compound, pharmaceutically acceptable salt, prodrug, active metabolite, or solvate) in conjunction with administration of temozolimide and/or radiotherapy.

O⁶-benzylguanine is known to one of skill in the art. To accentuate hematopoietic toxicity, endogenous activity of MGMT in stem cells (or tumor cells) can be inactivated by O⁶-benzylguanine (O⁶BG). The present invention also encompasses the combined use of temozolomide and O⁶-benzylguanine (O⁶BG) for treating cancer, using the above-described dosing Regimens and/or dosing schedules. O⁶BG can be administered either prior to, concomitantly with or after administration of temozolomide as described herein.

As described above in Tables 1 and 2, in certain dosing Regimens of the present invention, in particular Regimen Nos. 4, 8, 10, and 15, encompass administration of a growth factor in combination with temozolomide. According to a preferred embodiment, the growth factor is GM-CSF, G-CSF, IL-1, IL-3, IL-6, or erythropoietin. Non-limiting examples of growth factors include Epogen® (epoetin alfa), Procrit® (epoetin alfa), Neupogen® (filgrastim, a human G-CSF), Aranesp® (hyperglycosylated recombinant darbepoetin alfa), Neulasta® (also branded Neupopeg, pegylated recombinant filgrastim, pegfilgrastim), Albupoietin™ (a long-acting erythropoietin), and Albugranin™ (albumin G-CSF, a long-acting G-CSF). According to a more preferred embodiment, the growth factor is G-CSF.

As used herein, “GM-CSF” means a protein which (a) has an amino acid sequence that is substantially identical to the sequence of mature (i.e., lacking a signal peptide) human GM-CSF described by Lee et al., Proc. Natl. Acad. Sci. U.S.A., 82:4360 (1985) and (b) has biological activity that is common to native GM-CSF.

Substantial identity of amino acid sequences means that the sequences are identical or differ by one or more amino acid alterations (deletions, additions, substitutions) that do not substantially impair biological activity. Among the human GM-CSFs, nucleotide sequence and amino add heterogeneity have been observed. For example, both threonine and isoleucine have been observed at position 100 of human GM-CSF with respect to the N-terminal position of the amino acid sequence. Also, Schrimsher et al., Biochem. J., 247:195 (1987), have disclosed a human GM-CSF variant in which the methionine residue at position 80 has been replaced by an isoleucine residue. GM-CSF of other species such as mice and gibbons (which contain only 3 methionines) and rats are also contemplated by this invention. Recombinant GM-CSFs produced in prokaryotic expression systems may also contain an additional N-terminal methionine residue, as is well known in the art. Any GM-CSF meeting the substantial identity requirement is included, whether glycosylated (i.e., from natural sources or from a eukaryotic expression system) or unglycosylated (i.e., from a prokaryotic expression system or chemical synthesis).

GM-CSF for use in this invention can be obtained from natural sources (U.S. Pat. No. 4,438,032; Gasson et al., supra; Burgess et al., supra; Sparrow et al., Wu et al., supra). GM-CSF having substantially the same amino acid sequence and the activity of naturally occurring GM-CSF may be employed in the present invention. Complementary DNAs (cDNAs) for GM-CSF have been cloned and sequenced by a number of laboratories, e.g., Gough et al., Nature, 309:763 (1984) (mouse); Lee et al., Proc. Natl. Acad. Sci. USA, 82:4360 (1985) (human); Wong et al., Science, 228:810 (1985) (human and gibbon); Cantrell et al., Proc. Natl. Acad. Sci. USA, 82:6250 (1985) (human), Gough et al., Nature, 309:763 (1984) (mouse); Wong et al., Science, 228:810 (1985) (human and gibbon); Cantrell et al., Proc. Natl. Acad. Sci. U.S.A., 82:6250 (1985) (human).

GM-CSF can also be obtained from Immunex, Inc. of Seattle, Wash. and Schering-Plough Corporation of Kenilworth, N.J. and from Genzyme Corporation of Boston, Mass.

In an advantageous embodiment of the present invention, temozolomide can be administered according to the methods taught herein in combination with an anti-emetic agent. Palonosetron, Tropisetron, Ondansetron, Granisetron, Bemesetron or a combination of at least two of the foregoing, very selective acting substances are employed as 5HT₃-receptor-antagonists which serve as enti-emetics. In this respect it is preferred that the amount of active anti-emetic substance in one dosage unit amounts to 2 to 10 mg, an amount of 5 to 8 mg active substance in one dosage unit being especially preferred. A daily dosage comprises generally an amount of active substance of 2 to 20 mg, particularly preferred is an amount of active substance of 5 to 16 mg. An NK-1 antagonist (neurokinin-1 antagonist) such as aprepitant alone or in combination with a steroid such as dexamethasone can also be used with or without a 5HT₃-receptor antagonist in the methods of the present invention. If necessary, those skilled in the art also know how to vary the active substance in a dosage unit or the level of the daily dosage according to the requirements. The factors determining this, such as body weight, overall constitution, response to the treatment and the like will constantly be monitored by the artisan in order to be able to react accordingly and adjust the amount of active substance in a dosage unit or to adjust the daily dosage if necessary.

According to yet another embodiment, temozolomide is administered using the methods taught herein in combination with a farnesyl protein transferase inhibitor.

According to other embodiments, temozolomide can be administered with another antineoplastic agent. Non-limiting examples of other useful antineoplastic agents include Uracil Mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Paclitaxel, Mithramycin, Deoxycoformycin, Mitomycin-C, L-Asparaginase, Interferons, Etoposide, Teniposide 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Oxaliplatin (Eloxatin®), Iressa (gefinitib, Zd1839), XELODA® (capecitabine), Tarceva® (erlotinib), Azacitidine (5-Azacytidine; 5-AzaC), and mixtures thereof.

Temozolomide may be administred with other anti-cancer agents such as the ones disclosed in U.S. Pat. Nos. 5,824,346, 5,939,098, 5,942,247, 6,096,757, 6,251,886, 6,316,462, 6,333,333, 6,346,524, and 6,703,400, all of which are incorporated by reference.

### EXPERIMENTS

A series of experimental studies were conducted as described below.

Colony Formation Assays

As detailed below, DAOY human glioma cells (high MGMT level), A375 human melanoma cells (high MGMT level), and LOX human melanoma cells (low MGMT level) in in vitro colony formation assays were treated with different dosing schedules of TMZ. In brief, sub-confluent plates containing cells (DAOY, A375, or LOX) were trypsinized, then rinsed and suspended in appropriate culture medium before seeding in 6-well plates. Cells were incubated for 18-24 hours at 37° C. to allow cells to attach. Graded concentrations of TMZ or equivalent volumes of diluents were added in triplicate. Each pulse of TMZ lasted for 24 hours. For example, cells receiving continuous daily dosing of TMZ were treated with TMZ-containing medium every 24 hours throughout the cycle. Following the last pulse of TMZ in a cycle, TMZ-containing medium was removed and replaced with fresh medium without TMZ for the rest of the incubation period. Resulting colonies were stained with Crystal Violet solution and quantified using ImagePro plus software (Empire Imaging Systems, Inc. Asbury, N.J.).

DAOY Human Glioma Cell Line (High MGMT Level)

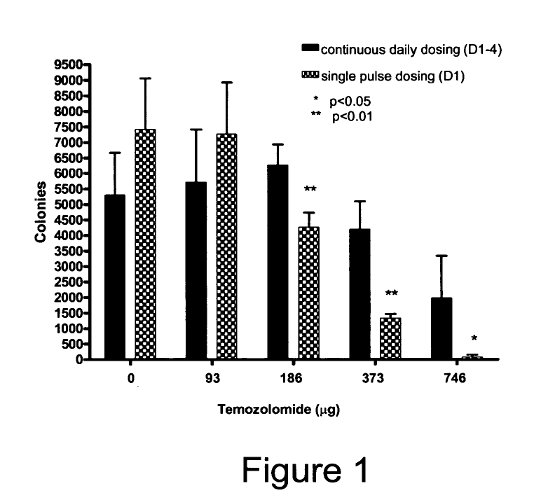

As illustrated in FIG. 1, colony formation assays were conducted whereby DAOY human glioma cells (high MGMT) were treated for a 4-day cycle according to one of two different TMZ dosing schedules: (i) continuous daily dosing (i.e., ¼ of total amount administered daily for four consecutive days; Day 1-4); or (ii) single pulse dosing (i.e., total amount administered in 1 day, Day 1); where the total amount of TMZ administered was 0, 93, 186, 373, or 746 μg. In short, single pulse dosing demonstrated better inhibition of colony formation than the continuous daily dosing at total TMZ levels of 186, 373, and 746 μg.

A375 Human Melanoma Cell Line (High MGMT Level)

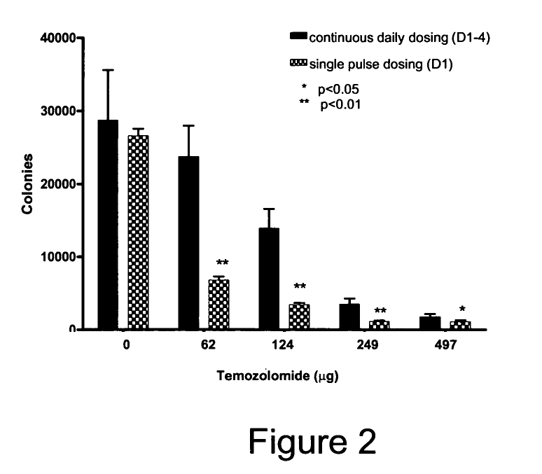

As illustrated in FIG. 2, colony formation assays were conducted whereby A375 human melanoma cells (high MGMT) were treated for a 4-day cycle according to one of two different TMZ dosing schedules: (i) continuous daily dosing (Day 1-4); or (ii) single pulse dosing (Day 1); where the total amount of TMZ administered was 0, 62, 124, 249, or 497 μg. Interestingly, a similar pattern of response was observed in A375 human melanoma cells as that in DAOY human glioma cells. Dose-dependent inhibition by TMZ was demonstrated using both TMZ dosing schedules, but single pulse dosing resulted in better inhibition of colony formation than continuous daily dosing at total TMZ levels of 62, 124, 249, 497 μg.

LOX Human Melanoma Cell Line (Low MGMT Level)

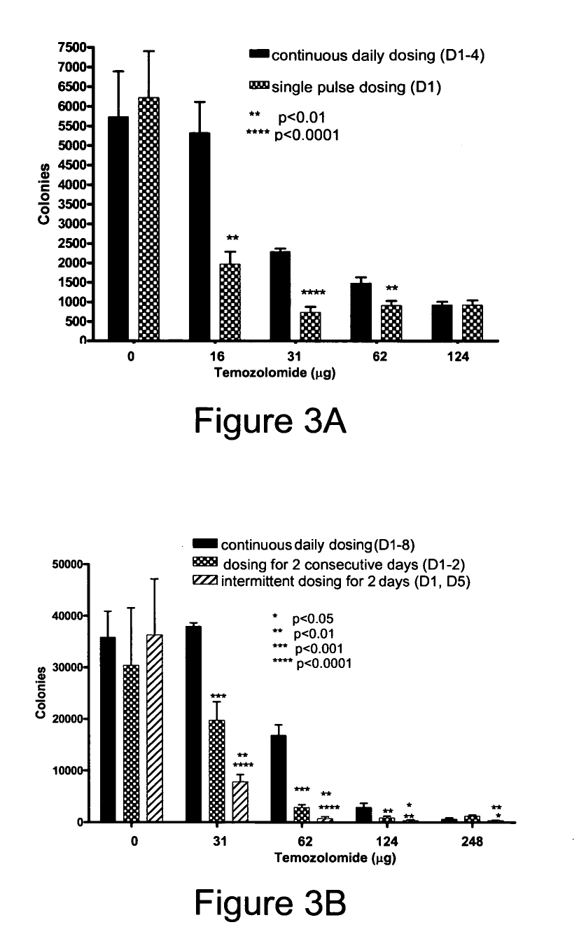

As illustrated in FIGS. 3A and 3B, colony formation assays were conducted whereby LOX human melanoma cells (low MGMT) were treated with TMZ dosing schedules for either a 4-day cycle (FIG. 3A) or an 8-day cycle (FIG. 3B).

In the 4-day cycle, illustrated in FIG. 3A, TMZ was administered according to one of two different dosing schedules: (i) continuous daily dosing (Day 1-4); or (ii) single pulse dosing (Day 1); where the total amount of TMZ administered was 0, 16, 31, 62, or 124 μg. Single pulse dosing demonstrated better inhibition of colony formation than continuous daily dosing.

In the 8-day cycle, illustrated in FIG. 3B, TMZ was administered according to one of three different dosing schedules: (i) continuous daily dosing (Day 1-8); (ii) dosing for 2 consecutive days (Day 1-2); or (ii) intermittent dosing for 2 days (Day 1, Day 5); where the total amount of TMZ administered was 0, 31, 62, 124, or 248 μg. Intermittent dosing for 2 days demonstrated better inhibition of colony formation than continuous daily dosing. In addition, intermittent dosing for 2 days demonstrated better inhibition of colony formation than dosing for 2 consecutive days at the same total TMZ dose.

MGMT Assays

As detailed below, the enzymatic activity and protein level of MGMT were determined in A375 human melanoma cells following TMZ treatment at different concentration levels (0, 10, 40, and 160 μM) for either: (i) 72 hours of TMZ treatment; or (ii) 72 hours of TMZ treatment followed by an additional 72 hours without TMZ treatment.

MGMT Enzymatic Activity Assay

In brief, ³H-methylated DNA substrate was prepared from calf thymus DNA. This substrate was incubated with 50 μg of cell extract at 37° C. for 45 min. After a complete transfer of radioactivity to MGMT protein, excess DNA was hydrolyzed and washed with trichloroacetic acid (TCA). Radioactivity transferred to MGMT protein was measured by scintillation counting.

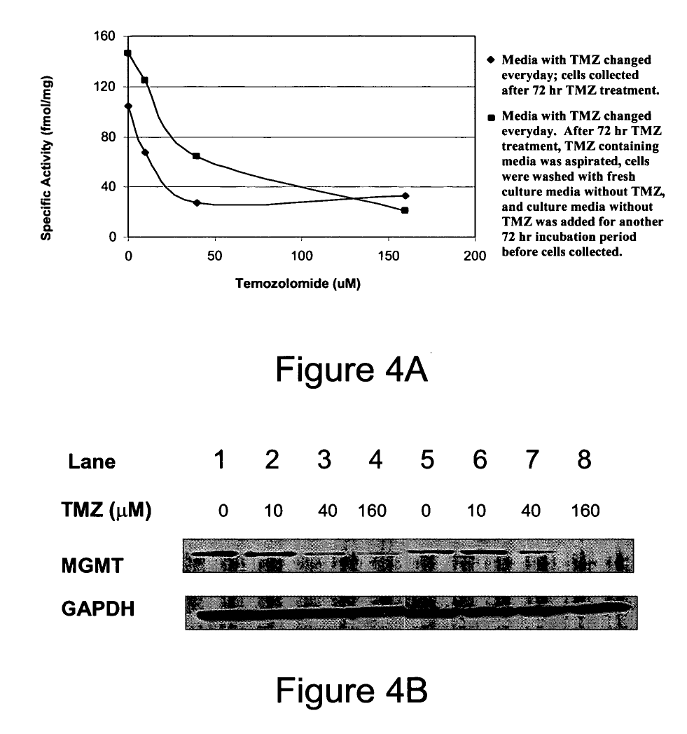

As illustrated in FIG. 4A, the level of MGMT enzymatic activity was measured in A375 melanoma cells following TMZ treatment at different concentration levels (0, 10, 40, and 160 mM). Treatment of TMZ for 72 hours caused dose-dependent reduction of MGMT. Moreover, to evaluate how long the reduction of MGMT activity persists after removal of drug treatment, enzyme activity was also measured in a parallel set of cells that, after the 72-hour treatment, were washed and maintained in medium without TMZ for another 72 hours. Interestingly, the enzyme activity remained reduced in a dose-dependent manner for 72 hours after drug removal. This indicates that high dose pulse treatment of TMZ has a prolonged effect on the level of MGMT, which also indicates that a subsequent dose of TMZ treatment of these cells may potentiate the cytotoxicity of TMZ.

MGMT Western Blot

Tumor cells (5×10⁵) were seeded in 100 mm×20 mm culture plates containing 10 ml of 90% DMEM (GIBCO, N.Y.) with 10% fetal bovine serum. Cells were treated with increasing concentrations of TMZ or equivalent volume of diluents. At various times after treatment, whole-cell lystes were prepared in a solution containing 10 mM Tris-HCl (pH7.5), 10 mM NaH₂PO₄/NaHPO₄, 130 mM NaCl, 1% Triton X-100, 10 mM PPi (BD Biosciences Pharmingen). Equal amounts of total protein were electrophoresed on a 4-12% SDS-polyacrylamide gel and electrotransferred to polyvinylidene defluoride membranes. The blots were blocked with 5% non-fat dry milk in Tris buffered saline (TBS) and probed with specific antibodies against MGMT (BD Bioscience Pharmingen) or against GAPDH (USBiological) as an internal control.

As illustrated in FIG. 4B, the level of MGMT protein was assayed by Western blot in A375 melanoma cells following TMZ treatment at different concentration levels (0, 10, 40, 160 μM). Lanes 1-4 reflect cell lysates prepared after 72 hours of TMZ treatment. Lanes 5-8 reflect cell lysates prepared after 72 hours of TMZ treatment followed by an additional 72 hours without TMZ treatment. The level of MGMT protein level detected correlated to the level of MGMT specific activity measured in similarly treated cells described in FIG. 4A. In both assays, a dose-dependent reduction in MGMT protein level was detected.

In vivo Studies

As detailed below, different TMZ dosing schedules were evaluated in xenograft tumors formed using DAOY human glioma cells (high MGMT level), A375 human melanoma cells (high MGMT level), and LOX human melanoma cells (low MGMT level).

In brief, female athymic nude mice or female SCID mice (4-6 week old) from Charles River Laboratories were maintained in a VAF-barrier facility. Animal procedures were performed in accordance with the rules set forth in the N.I.H. guide for the care and use of laboratory animals.

DAOY human glioma cells (5×10⁶), LOX human melanoma cells (5×10⁵), and A375 human melanoma cells (5×10⁶) were inoculated subcutaneously in the right flank of the animal (LOX in SCID mice; DAOY and A375 in nude mice). To facilitate in vivo growth, Matrigel was mixed with DAOY and A375 cells (50%) before inoculation. When tumor volumes were approximately 100 mm³, animals were randomized and grouped (n=10). Tumor volumes and body weight were measured twice weekly using Labcat™ computer application (Innovative Programing Associates, N.J.). Tumor volumes were calculated by the formula (W×L×H)×π×⅙.

TMZ was administered by intraperitoneal injections with 20% HPβCD (containing 1% DMSO) as vehicle. Mice bearing xenograft tumors of DAOY human glioma cells, a high MGMT level cell line, were treated with one of three different dosing schedules. In a 15-day cycle, under same total dose levels, mice received one of the following TMZ treatments: (i) day 1 through day 15; (ii) day 1 through day 5; or (iii) intermittently on day 1, 4, 7, 10, and 13. For all dosing schedules, three different dose levels (180, 270, and 405 mg/kg total) were used.

Mice bearing xenograft tumors of A375 human melanoma cells, a high MGMT level cell line, were treated with three different dosing schedules. Similar to the schedules used for the DAOY model, in a 15-day cycle, under same total dose levels, mice received one of the following TMZ treatments: (i) day 1 through day 15; (ii) day 1 through day 5; or (iii) intermittently on day 1, 4, 7, 10, and 13. For all dosing schedules, three different dose levels (180, 270, and 405 mg/kg total) were used.

Mice bearing xenograft tumors of LOX human melanoma cells, a low MGMT level cell line, were treated with TMZ using two different schedules. The same total dose was administered evenly divided over the course of either: (i) 4 or (ii) 12 days. TMZ was administered through intraperitoneal injection using cumulative total dose levels of 36, 72 or 144 mg/kg.

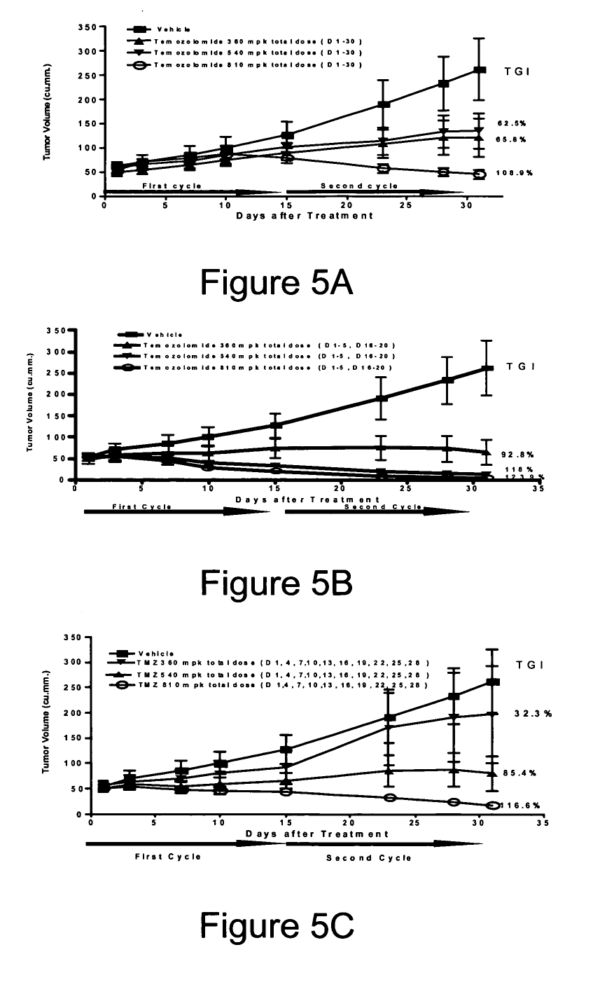

As illustrated in FIG. 5, nude mice bearing xenograft tumors of DAOY human glioma cells, a high MGMT level cell line, were treated with three different schedules of TMZ in a dose-dependent fashion. FIG. 5A illustrates the mean tumor growth curves of DAOY human glioma xenograft tumors following TMZ treatment for two consecutive 15-day cycles of continuous daily dosing (Day 1-15 (first cycle), Day 16-30 (second cycle)); where the total dose of TMZ administered was 0, 360, 540, or 810 mg per kg (mpk). FIG. 5B illustrates the mean tumor growth curves of DAOY human glioma xenograft tumors following TMZ treatment for two consecutive 15-day cycles of dosing for 5 consecutive days (Day 1-5 (first cycle); Day 16-20 (second cycle)); where the total dose of TMZ administered was 0, 360, 540, or 810 mpk. FIG. 5C illustrates mean tumor growth curves of DAOY human glioma xenograft tumors following TMZ treatment for two consecutive 15-day cycles of intermittent dosing for 5 days (Day 1, 4, 7, 10, 13 (first cycle); Day 16, 19, 22, 25, 28 (second cycle)); where the total dose of TMZ administered was 0, 360, 540, or 810 mpk. Notably, the mean tumor volume of each treatment group during the period of therapy is represented. In this tumor model, both the dosing for 5 consecutive days and the intermittent dosing for five days demonstrated better tumor growth inhibition than the continuous daily dosing schedule (Day 1-15). In fact, tumor regression occurred after merely one cycle of treatment with either the two higher dose levels of TMZ (54 or 81 mg/kg/day) in the dosing for 5 consecutive days as well as with the highest dose level of TMZ (81 mg/kg/day) in the intermittent dosing schedule.

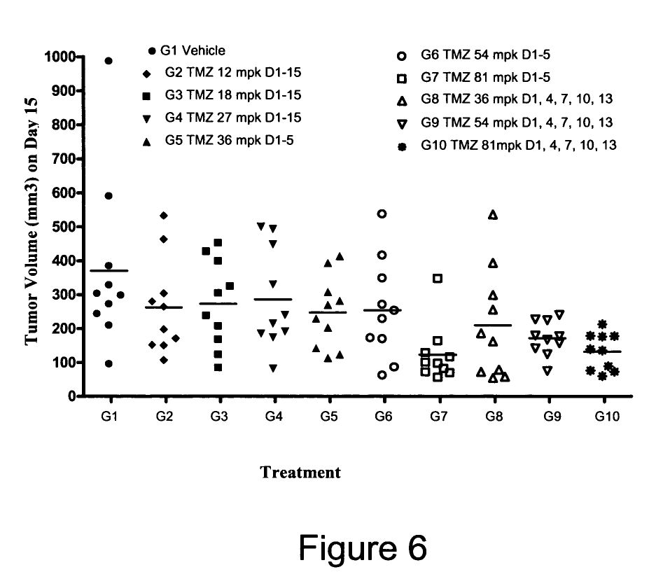

As illustrated in FIG. 6, nude mice bearing xenograft tumors of A375 human melanoma cells, a high MGMT cell line, were treated with the same dosing schedules as were mice in the DAOY human glioma xenograft tumor study discussed above. A similar pattern was observed in A375 human melanoma xenograft tumors as those of DAOY glioma xenograft tumors. Notably, the two higher dose levels of intermittent dosing schedule (Day 1, 4, 7, 10, 13) and the highest dose level of the dosing for 5 consecutive days (Day 1-5) generated significantly better efficacy than the equivalent dose levels of the continuous daily dosing schedule (Day 1-15).

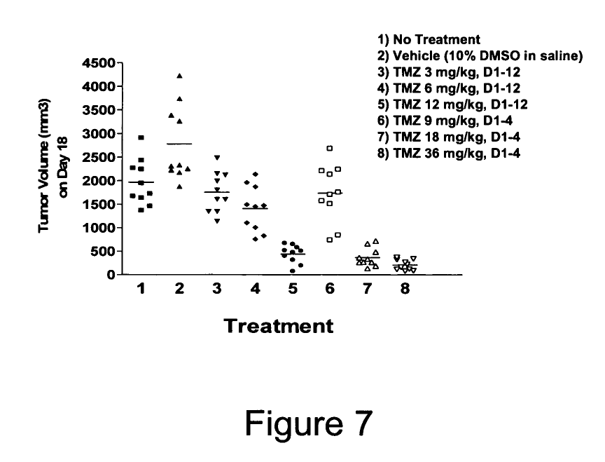

As illustrated in FIG. 7, SCID mice bearing xenograft tumors of LOX melanoma cells, a low MGMT cell line, were treated with two different dosing schedules for a 12-day cycle: (i) dosing for 4 consecutive days (Day 1-4); or (ii) continuous daily dosing (Day 1-12). At the intermediate dose (72 mg/kg), the 4-day treatment schedule induced significantly better efficacy (88% TGI) than the 12-day schedule (50% TGI). In contrast, no statistical difference was observed at higher and lower dose levels. The efficacy of TMZ was schedule dependent, with greater efficacy seen when dosing for 4 consecutive days.

Intratumoral MGMT Enzymatic Activity

In brief, three DAOY tumors treated with either 81 mg/kg TMZ or vehicle for five consecutive days were collected from mice. Each tumor was homogenized and processed for MGMT enzymatic activity following treatment. MGMT activity measured from untreated DAOY cells was also included as a control.

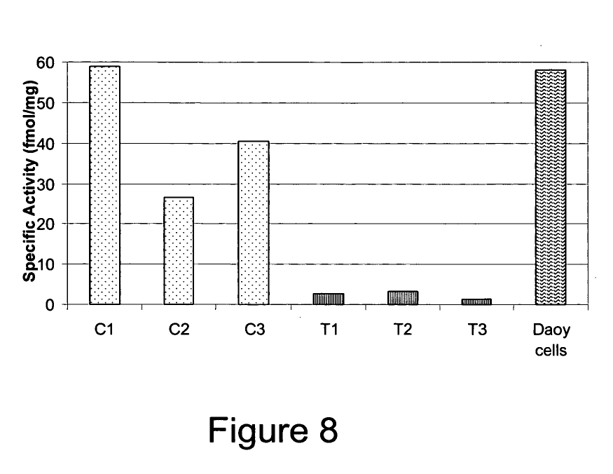

As illustrated in FIG. 8, unlike tumors treated with vehicle which had similar level of MGMT activity compared to DAOY cells harvested from cell culture, tumors that had been treated for five consecutive days with TMZ had little MGMT activity detected.

Summary

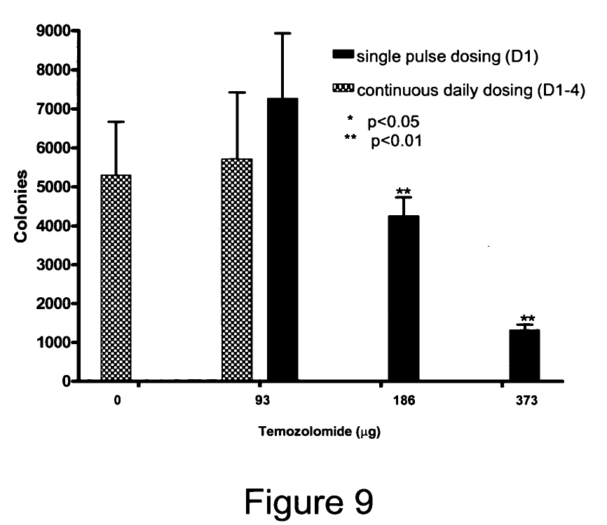

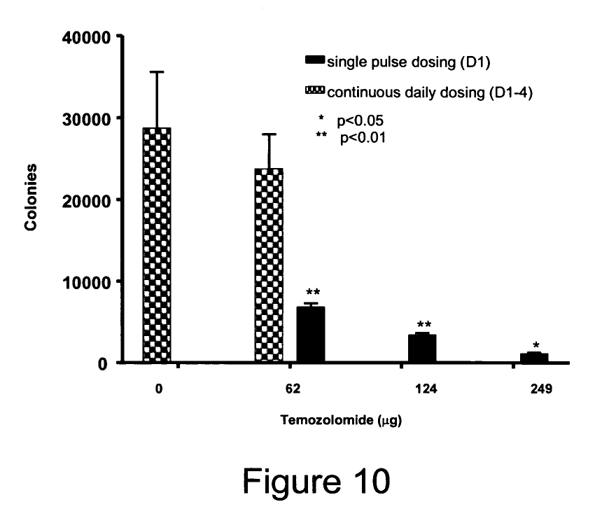

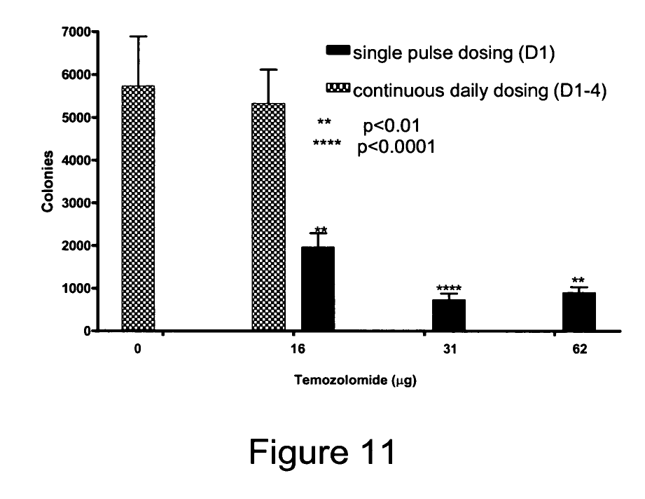

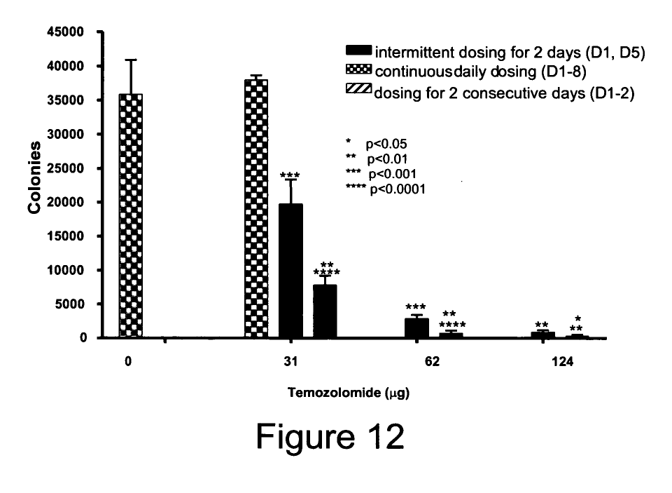

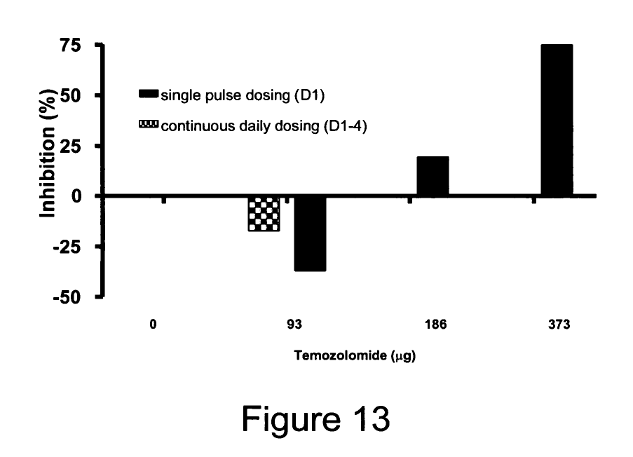

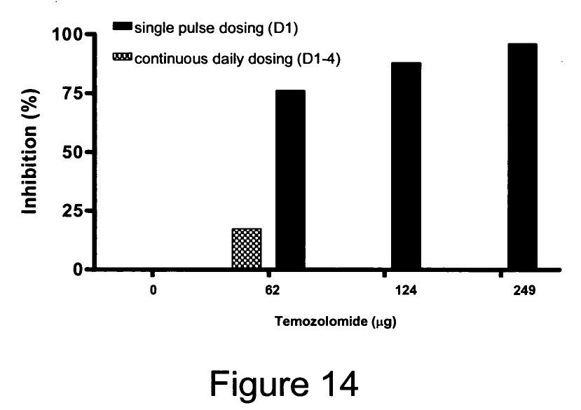

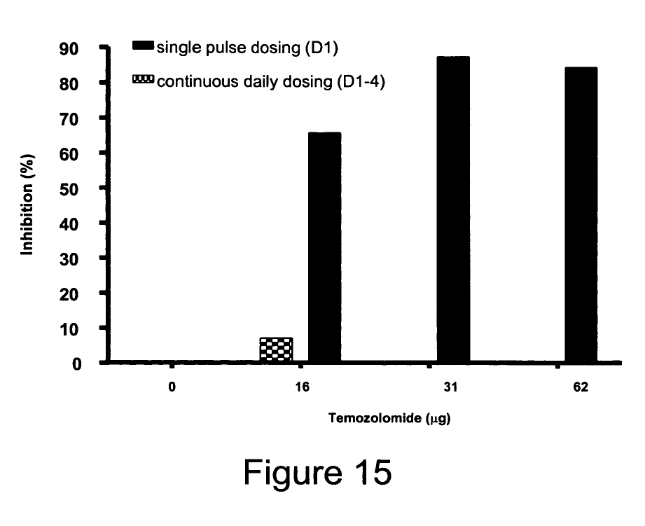

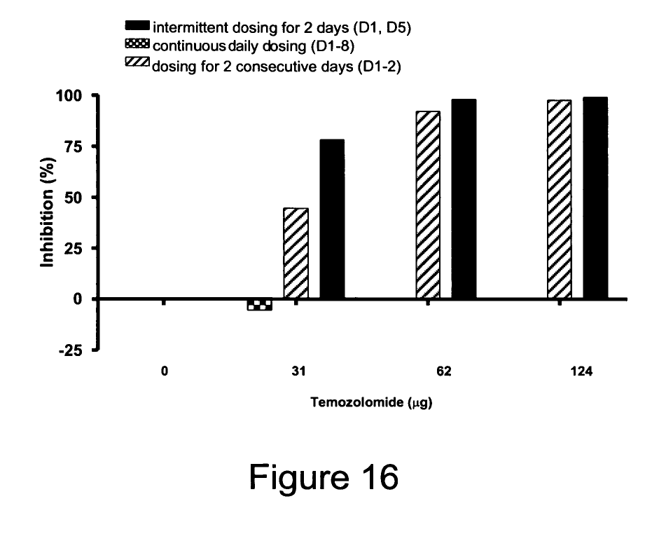

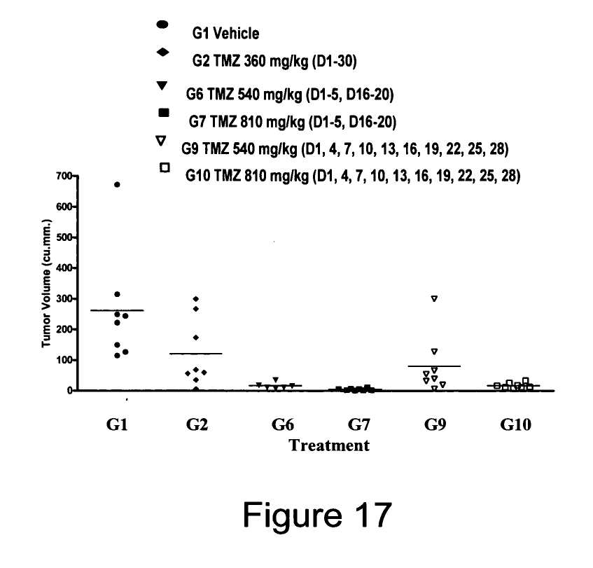

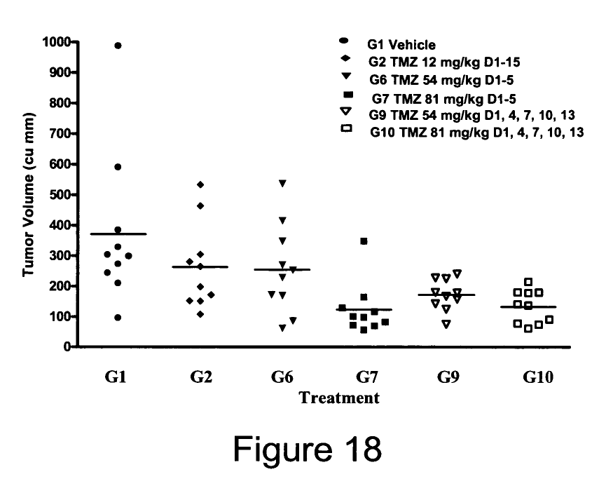

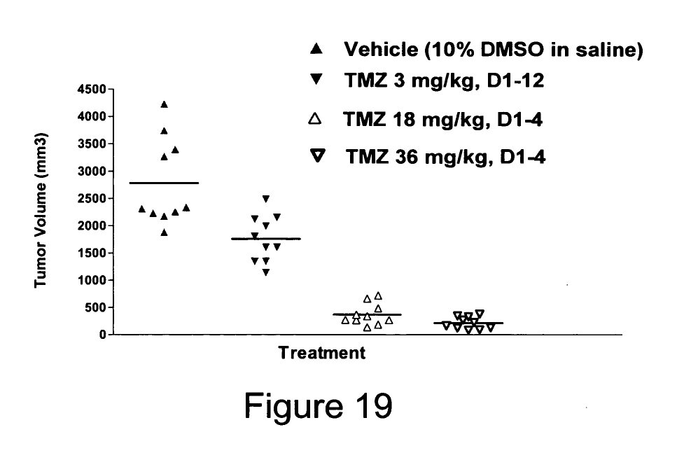

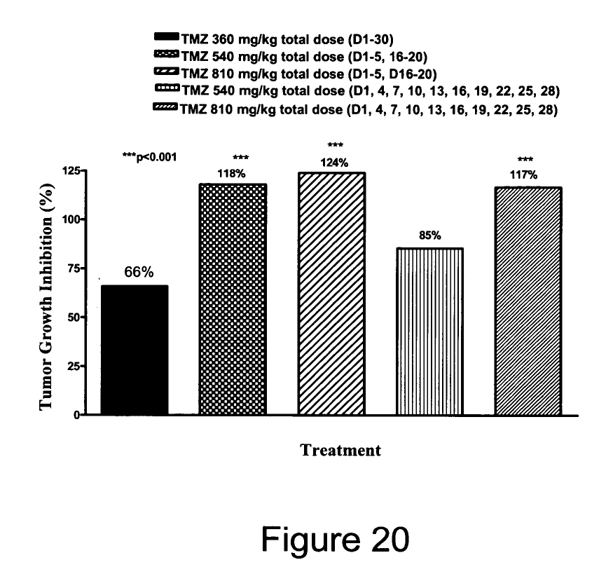

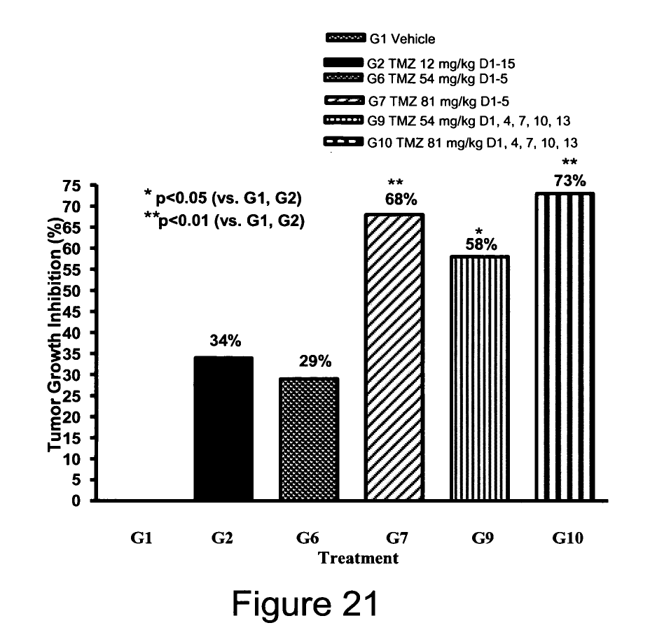

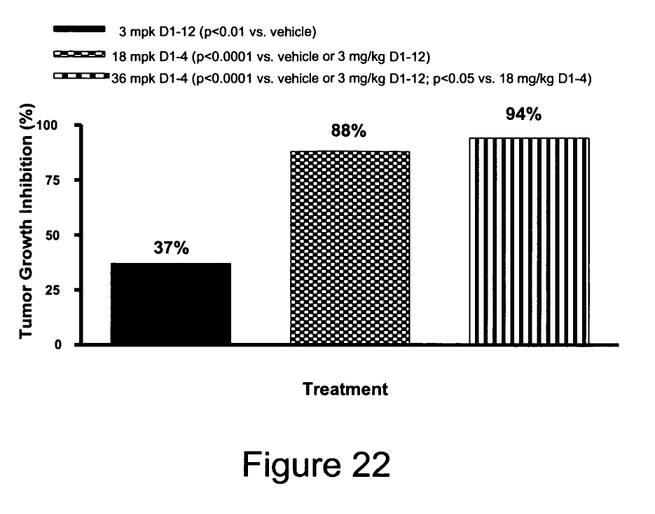

A subset of data obtained from the aforementioned experiments is recharted in FIGS. 9-22 for illustrative purposes. In addition, Tables 3-5 below summarize the same subset of data.

TABLE 3
Colony Formation Assays

| | | | Total | % Inhibition |
|---|---|---|---|---|
| Cell | | | TMZ | compared to |
| type | Dosing schedule | | (μg) | untreated cells |
| DAOY | 4-day cycle - | Day 1-4 | 93 | −8 |
| | continuous | (¼ total TMZ/day) | | |
| | daily dosing | | | |
| DAOY | 4-day cycle - | Day 1 | 93 | −37 |
| | single pulse dosing | | | |
| DAOY | 4-day cycle - | Day 1 | 186 | 20 |
| | single pulse dosing | | | |
| DAOY | 4-day cycle - | Day 1 | 373 | 75 |
| | single pulse dosing | | | |
| A375 | 4-day cycle - | Day 1-4 | 62 | 17.4 |
| | continuous | (¼ total TMZ/day) | | |
| | daily dosing | | | |
| A375 | 4-day cycle - | Day 1 | 62 | 76 |
| | single pulse dosing | | | |
| A375 | 4-day cycle - | Day 1 | 124 | 88 |
| | single pulse dosing | | | |
| A375 | 4-day cycle - | Day 1 | 249 | 96 |
| | single pulse dosing | | | |
| LOX | 4-day cycle - | Day 1-4 | 16 | 7 |
| | continuous | (¼ total TMZ/day) | | |
| | daily dosing | | | |
| LOX | 4-day cycle - | Day 1 | 16 | 66 |
| | single pulse dosing | | | |
| LOX | 4-day cycle - | Day 1 | 31 | 87 |
| | single pulse dosing | | | |
| LOX | 4-day cycle - | Day 1 | 62 | 84 |
| | single pulse dosing | | | |
| LOX | 8-day cycle - | Day 1-8 | 31 | −5 |
| | continuous | (⅛ total TMZ/day) | | |
| | daily dosing | | | |
| LOX | 8-day cycle - | Day 1, Day 2 (½ total | 31 | 45 |
| | dosing for 2 consecutive | TMZ/day) | | |
| | days | | | |
| LOX | 8-day cycle - | Day 1, Day 2 (½ total | 62 | 92 |
| | dosing for 2 consecutive | TMZ/day) | | |
| | days | | | |
| LOX | 8-day cycle - | Day 1, Day 2 (½ total | 124 | 98 |
| | dosing for 2 consecutive | TMZ/day) | | |
| | days | | | |
| LOX | 8-day cycle - | Day 1, Day 5 (½ total | 31 | 78 |
| | intermittent dosing for 2 | TMZ/day) | | |
| | days | | | |
| LOX | 8-day cycle - | Day 1, Day 5 (½ total | 62 | 98 |
| | intermittent | TMZ/day) | | |
| | dosing for 2 days | | | |
| LOX | 8-day cycle - | Day 1, Day 5 (½ total | 124 | 99 |
| | intermittent dosing for 2 | TMZ/day) | | |
| | days | | | |

TABLE 4
DAOY Human Glioma Xenograft Tumor Model

| | | | TMZ | | | |
|---|---|---|---|---|---|---|
| | | | dose | Total | | % Inhibition |
| | | | per | TMZ | | compared to |
| Cell | | | pulse | dose | Dose | tumor treated |
| type | Dosing schedule | | (mpk) | (mpk) | Intensity | with vehicle |
| DAOY | Two 15-day cycles - | Day 1-30 | 12 | 360 | 1 | 66 |
| | continuous | (1/30 total | | | | |
| | dosing | dose/day) | | | | |
| DAOY | Two 15-day cycles - | Day 1-5, | 54 | 540 | 1.5 | 118 |
| | dosing for 5 | Day 16-20 | | | | |
| | consecutive days | (1/10 total | | | | |
| | | dose/day) | | | | |
| DAOY | Two 15-day cycles - | Day 1-5, | 81 | 810 | 2.25 | 124 |
| | dosing for 5 | Day 16-20 | | | | |
| | consecutive days | (1/10 total | | | | |
| | | dose/day) | | | | |
| DAOY | Two 15-day cycles - | Day 1, 4, 7, | 54 | 540 | 1.5 | 85 |
| | intermittent dosing | 10, 13, 16, | | | | |
| | for 5 days | 19, 22, 25, 28 | | | | |
| | | (1/10 total | | | | |
| | | dose/day) | | | | |
| DAOY | Two 15-day cycles - | Day 1, 4, 7, | 81 | 810 | 2.25 | 117 |
| | intermittent dosing | 10, 13, 16, | | | | |
| | for 5 days | 19, 22, 25, 28 | | | | |
| | | (1/10 total | | | | |
| | | dose/day) | | | | |

TABLE 5
A375 and LOX Human Melanoma Xenograft Tumor Models

| | | | TMZ | | | % Inhibition |
|---|---|---|---|---|---|---|
| | | | dose | Total | | compared |
| | | | per | TMZ | | to tumor |
| Cell | | | pulse | dose | | treated with |
| type | Dosing Schedule | | (mpk) | (mpk) | Dose Intensity | vehicle |
| A375 | 15-day cycle - | Day 1-15 | 12 | 180 | 1 | 34 |
| | continuous daily dosing | (1/15 total | | | | |
| | | dose/day) | | | | |
| A375 | 15-day cycle - | Day 1-5 | 54 | 270 | 1.5 | 29 |
| | dosing for 5 consecutive days | (1/5 total | | | | |
| | | dose/day) | | | | |
| A375 | 15-day cycle - | Day 1-5 | 81 | 405 | 2.25 | 68 |
| | dosing for 5 consecutive days | (1/5 total | | | | |
| | | dose/day) | | | | |
| A375 | 15-day cycle - | Day 1, 4, | 54 | 270 | 1.5 | 58 |
| | intermittent dosing for 5 days | 7, 10, 13 | | | | |
| | | (1/5 total | | | | |
| | | dose/day) | | | | |
| A375 | 15-day cycle - | Day 1, 4, | 81 | 405 | 2.25 | 73 |
| | intermittent dosing for 5 days | 7, 10, 13 | | | | |
| | | (1/5 total | | | | |
| | | dose/day) | | | | |
| LOX | 12-day cycle - | Day 1-12 | 3 | 36 | 1 | 37 |
| | continuous daily dosing | (1/12 total | | | | |
| | | dose/day) | | | | |
| LOX | 12-day cycle - | Day 1-4 | 18 | 72 | 2 | 88 |
| | dosing for 4 consecutive days | (1/4 total | | | | |
| | | dose/day) | | | | |
| LOX | 12-day cycle - | Day 1-4 | 36 | 144 | 4 | 94 |
| | dosing for 4 consecutive days | (1/4 total | | | | |
| | | dose/day) | | | | |

The inventors conclude that these studies demonstrate dosing schedules with increased total TMZ dose as examined in xenograft tumor models are more efficacious at inhibiting cell growth in tumor cell lines with a high level of MGMT. In particular, dosing schedules of TMZ that correlate with enhanced dose intensity (i.e., greater than a Dose Intensity of 1) are more efficacious than those of a standard dose intensity (i.e., Dose Intensity of 1) at inhibiting tumor cell growth in xenografts derived from cell lines with a high level of MGMT.

Although certain presently preferred embodiments of the invention have been described herein, it will be apparent to those skilled in the art to which the invention pertains that variations and modifications of the described embodiments may be made without departing from the spirit and scope of the invention. Accordingly, it is intended that the invention be limited only to the extent required by the appended claims.

We claim:

1. A method for treating a patient having a glioma, comprising: administering to the patient, temozolomide either:
I. according to the following regimen: 150-200 mg/m² per day for 5 days in a 28 day cycle if methylation of the MGMT gene is detected, in a sample obtained from the patient, using methylation-specific PCR (MSP); or
II. according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is not detected, in a sample obtained from the patient, using MSP.

2. A method for treating a patient having a glioma, comprising:
I. assessing whether the MGMT gene is methylated, in a sample obtained from the patient, using MSP; and
II. administering temozolomide to the patient either:
a) according to the following regimen: at 150-200 mg/m² per day for 5 days in a 28 day cycle if methylation of the MGMT gene is detected in the sample; or
b) according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is not detected in the sample.

3. The method of claim 2, in which the sample is a tumor biopsy sample.

4. A method of treating a patient having a glioma, comprising: administering temozolomide to the patient either:
a) according to the following regimen: 150-200 mg/m² per day for 5 days in a 28 day cycle if the MGMT protein is not detected, in a sample obtained from the patient using a Western blot immunoassay, an immunohistochemical technique, or an enzymatic assay for MGMT protein; or
b) according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is detected in a sample obtained from the patient using a Western blot immunoassay, an immunohistochemical technique, or an enzymatic assay for MGMT protein.

5. A method for treating a patient having a glioma, comprising:
I. conducting a Western blot, an immunohistochemical assay or an enzymatic assay to detect the presence or absence of the MGMT protein in a sample obtained from the patient; and
II. administering temozolomide to the patient either:
a) according to the following regimen: at 150-200 mg/m² per day for 5 days in a 28 day cycle if the MGMT protein is not detected in the sample; or
b) according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is present in the sample.

6. The method of claim 5, in which the sample is a tumor biopsy sample.

7. A method of treating a patient having a glioma, comprising: administering temozolomide, to the patient as follows:
a) according to either of the following two regimens:
i. 150-200 mg/m² per day for 5 days in a 28 day cycle; or
ii. 250 mg/m² per day for 5 days in a 28 day cycle in combination with a growth factor,
if the level or enzymatic activity of the MGMT protein detected in a sample obtained from the patient is Low, compared to that of normal lymphocytes; or
b) according to one of the following four regimens if the level or enzymatic activity of the MGMT protein detected in a sample obtained from the patient is Moderate, compared to that of normal lymphocytes:
i. 100 mg/m² per day for 14 days in a 28 day cycle; or
ii. 300 mg/m² per day for 5 days in a 28 day cycle in combination with a growth factor; or
iii. 75 mg/m² per day for 21 days in a 28 day cycle; or
iv. 75 mg/m² per 42 days in a 56 day cycle; or
c) according to one of the following three regimens if the level or enzymatic activity of the MGMT protein detected in a sample obtained from the patient is High, compared to that of normal lymphocytes:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

8. The method of claim 7, in which the sample is a tumor biopsy sample.

9. A method for treating a patient having a glioma, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient as follows:
a) if the patient's level or enzymatic activity of the MGMT protein is classified in step I as Low, compared to that of normal lymphocytes, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's level or enzymatic activity of the MGMT protein is classified as Moderate compared to that of normal lymphocytes, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's level or enzymatic activity of the MGMT protein is classified as High, compared to that of normal lymphocytes, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

10. A method for treating a patient having a glioma, comprising:
I. assessing the level of methylation of the MGMT gene in a sample obtained from the patient and classifying the level as Low, Moderate, or High as compared to that of normal lymphocytes; and
II. administering temozolomide to the patient as follows:
a) if the patients' level of methylation of the MGMT gene is classified as Low compared to that of normal lymphocytes, according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle if methylation of the MGMT gene is not detected in the sample; or
b) if the patient's level of methylation of the MGMT gene is classified as Moderate compared to that of normal lymphocytes, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² per day for 21 days in a 28 day cycle; or
iv. 75 mg/m² per 42 days in a 56 day cycle; or
c) if the patient's level of methylation of the MGMT gene is classified as High as compared to that of normal lymphocytes, according to one of the following two regimens:
i. 150-200 mg/m² per day for 5 days in a 28 day cycle; or
ii. 250 mg/m² per day for 5 days in a 28 day cycle in combination with a growth factor.

11. A method for treating a patient having a melanoma, comprising administering to the patient, temozolomide according to one of the following regimens:
I. 100 mg/m² per day for 14 days in a 21 day cycle; or
II. 150 mg/m² per day for 7 days in a 14 day cycle; or
III. 100 mg/m² per day for 21 days in a 28 day cycle
if methylation of the MGMT gene is detected, in a sample obtained from the patient, using MSP.

12. A method for treating a patient having a melanoma, comprising:
I. assessing whether the MGMT gene is methylated, in a sample obtained from the patient, using MSP; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is detected in the sample.

13. The method of claim 12, in which the sample is a tumor biopsy sample.

14. A method of treating a patient having a melanoma, comprising administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is not detected in a sample obtained from the patient using a Western blot immunoassay, an immunohistochemical technique, or an enzymatic assay for MGMT protein.

15. A method for treating a patient having a melanoma, comprising:
I. conducting a Western blot, an immunohistochemical assay or an enzymatic assay to detect the presence or absence of the MGMT protein in a sample obtained from the patient; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if MGMT protein is not detected in the sample.

16. The method of claim 5, in which the sample is a tumor biopsy sample.

17. A method of treating a patient having a melanoma, comprising administering temozolomide to the patient according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the level of the MGMT protein or enzymatic activity detected in a sample obtained from the patient is Low or Moderate, compared to that of normal lymphocytes.

18. The method of claim 17, in which the sample is a tumor biopsy sample.

19. A method for treating a patient having a melanoma, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patient's level or enzymatic activity of the MGMT protein is classified as Low or Moderate, compared to that of normal lymphocytes.

20. A method for treating a patient having a melanoma, comprising:
I. assessing the level of methylation of the MGMT gene in a sample obtained from the patient and classifying the level as Low, Moderate, or High as compared to that of normal lymphocytes; and
II. administering temozolomide to the patient according to one of the regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patients' level of methylation of the MGMT gene is classified as Moderate or High compared to that of normal lymphocytes.

21. A method for treating a patient having a lung cancer, comprising administering to the patient, temozolomide according to one of the following regimens:
I. 100 mg/m² per day for 14 days in a 21 day cycle; or
II. 150 mg/m² per day for 7 days in a 14 day cycle; or
III. 100 mg/m² per day for 21 days in a 28 day cycle
if methylation of the MGMT gene is detected, in a sample obtained from the patient, using MSP.

22. A method for treating a patient having a lung cancer, comprising:
I. assessing whether the MGMT gene is methylated, in a sample obtained from the patient, using MSP; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is detected in the sample.

23. The method of claim 12, in which the sample is a tumor biopsy sample.

24. A method of treating a patient having a lung cancer, comprising administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is not detected in a sample obtained from the patient using a Western blot immunoassay, an immunohistochemical technique, or an enzymatic assay for MGMT protein.

25. A method for treating a patient having a lung cancer, comprising:
I. conducting a Western blot, an immunohistochemical assay or an enzymatic assay to detect the presence or absence of the MGMT protein in a sample obtained from the patient; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if MGMT protein is not detected in the sample.

26. The method of claim 5, in which the sample is a tumor biopsy sample.

27. A method of treating a patient having a lung cancer, comprising administering temozolomide to the patient according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the level of the MGMT protein or enzymatic activity detected in a sample obtained from the patient is Low or Moderate, compared to that of normal lymphocytes.

28. The method of claim 17, in which the sample is a tumor biopsy sample.

29. A method for treating a patient having a lung cancer, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patient's level or enzymatic activity of the MGMT protein is classified as Low or Moderate, compared to that of normal lymphocytes.

30. A method for treating a patient having a lung cancer, comprising:
I. assessing the level of methylation of the MGMT gene in a sample obtained from the patient and classifying the level as Low, Moderate, or High as compared to that of normal lymphocytes; and
II. administering temozolomide to the patient according to one of the regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patients' level of methylation of the MGMT gene is classified as Moderate or High compared to that of normal lymphocytes.

31. A method for treating a patient having a lymphoma, comprising administering to the patient, temozolomide according to one of the following regimens:
I. 100 mg/m² per day for 14 days in a 21 day cycle; or
II. 150 mg/m² per day for 7 days in a 14 day cycle; or
III. 100 mg/m² per day for 21 days in a 28 day cycle
if methylation of the MGMT gene is detected, in a sample obtained from the patient, using MSP.

32. A method for treating a patient having a lymphoma, comprising:
I. assessing whether the MGMT gene is methylated, in a sample obtained from the patient, using MSP; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is detected in the sample.

33. The method of claim 12, in which the sample is a tumor biopsy sample.

34. A method of treating a patient having a lymphoma, comprising administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is not detected in a sample obtained from 25 the patient using a Western blot immunoassay, an immunohistochemical technique, or an enzymatic assay for MGMT protein.

35. A method for treating a patient having a lymphoma, comprising:
I. conducting a Western blot, an immunohistochemical assay or an enzymatic assay to detect the presence or absence of the MGMT protein in a sample obtained from the patient; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if MGMT protein is not detected in the sample.

36. The method of claim 5, in which the sample is a tumor biopsy sample.

37. A method of treating a patient having a lymphoma, comprising administering temozolomide to the patient according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the level of the MGMT protein or enzymatic activity detected in a sample obtained from the patient is Low or Moderate, compared to that of normal lymphocytes.

38. The method of claim 17, in which the sample is a tumor biopsy sample.

39. A method for treating a patient having a lymphoma, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patient's level or enzymatic activity of the MGMT protein is classified as Low or Moderate, compared to that of normal lymphocytes.

40. A method for treating a patient having a lymphoma, comprising:
I. assessing the level of methylation of the MGMT gene in a sample obtained from the patient and classifying the level as Low, Moderate, or High as compared to that of normal lymphocytes; and
II. administering temozolomide to the patient according to one of the regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patients' level of methylation of the MGMT gene is classified as Moderate or High compared to that of normal lymphocytes.

41. A method for treating a patient having head and neck cancer, comprising administering to the patient, temozolomide according to one of the following regimens:
I. 100 mg/m² per day for 14 days in a 21 day cycle; or
II. 150 mg/m² per day for 7 days in a 14 day cycle; or
III. 100 mg/m² per day for 21 days in a 28 day cycle
if methylation of the MGMT gene is detected, in a sample obtained from the patient, using MSP.

42. A method for treating a patient having head and neck cancer, comprising:
I. assessing whether the MGMT gene is methylated, in a sample obtained from the patient, using MSP; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is detected in the sample.

43. The method of claim 12, in which the sample is a tumor biopsy sample.

44. A method of treating a patient having head and neck cancer, comprising administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is not detected in a sample obtained from the patient using a Western blot immunoassay, an immunohistochemical technique, or an enzymatic assay for MGMT protein.

45. A method for treating a patient having head and neck cancer, comprising:
I. conducting a Western blot, an immunohistochemical assay or an enzymatic assay to detect the presence or absence of the MGMT protein in a sample obtained from the patient; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if MGMT protein is not detected in the sample.

46. The method of claim 5, in which the sample is a tumor biopsy sample.

47. A method of treating a patient having head and neck cancer, comprising administering temozolomide to the patient according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the level of the MGMT protein or enzymatic activity detected in a sample obtained from the patient is Low or Moderate, compared to that of normal lymphocytes.

48. The method of claim 17, in which the sample is a tumor biopsy sample.

49. A method for treating a patient having head and neck cancer, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patient's level or enzymatic activity of the MGMT protein is classified as Low or Moderate, compared to that of normal lymphocytes.

50. A method for treating a patient having head and neck cancer, comprising:
I. assessing the level of methylation of the MGMT gene in a sample obtained from the patient and classifying the level as Low, Moderate, or High as compared to that of normal lymphocytes; and
II. administering temozolomide to the patient according to one of the regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patients' level of methylation of the MGMT gene is classified as Moderate or High compared to that of normal lymphocytes.

51. A method for treating a patient having ovarian cancer, comprising administering to the patient, temozolomide according to one of the following regimens:
I. 100 mg/m² per day for 14 days in a 21 day cycle; or
II. 150 mg/m² per day for 7 days in a 14 day cycle; or
III. 100 mg/m² per day for 21 days in a 28 day cycle
if methylation of the MGMT gene is detected, in a sample obtained from the patient, using MSP.

52. A method for treating a patient having ovarian cancer, comprising:
I. assessing whether the MGMT gene is methylated, in a sample obtained from the patient, using MSP; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is detected in the sample.

53. The method of claim 12, in which the sample is a tumor biopsy sample.

54. A method of treating a patient having ovarian cancer, comprising administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is not detected in a sample obtained from the patient using a Western blot immunoassay, an immunohistochemical technique, or an enzymatic assay for MGMT protein.

55. A method for treating a patient having ovarian cancer, comprising:
I. conducting a Western blot, an immunohistochemical assay or an enzymatic assay to detect the presence or absence of the MGMT protein in a sample obtained from the patient; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if MGMT protein is not detected in the sample.

56. The method of claim 5, in which the sample is a tumor biopsy sample.

57. A method of treating a patient having ovarian cancer, comprising administering temozolomide to the patient according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the level of the MGMT protein or enzymatic activity detected in a sample obtained from the patient is Low or Moderate, compared to that of normal lymphocytes.

58. The method of claim 17, in which the sample is a tumor biopsy sample.

59. A method for treating a patient having ovarian cancer, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patient's level or enzymatic activity of the MGMT protein is classified as Low or Moderate, compared to that of normal lymphocytes.

60. A method for treating a patient having ovarian cancer, comprising:
I. assessing the level of methylation of the MGMT gene in a sample obtained from the patient and classifying the level as Low, Moderate, or High as compared to that of normal lymphocytes; and
II. administering temozolomide to the patient according to one of the regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patients' level of methylation of the MGMT gene is classified as Moderate or High compared to that of normal lymphocytes.

61. A method for treating a patient having colorectal and/or colon cancer, comprising administering to the patient, temozolomide according to one of the following regimens:
I. 100 mg/m² per day for 14 days in a 21 day cycle; or
II. 150 mg/m² per day for 7 days in a 14 day cycle; or
III. 100 mg/m² per day for 21 days in a 28 day cycle
if methylation of the MGMT gene is detected, in a sample obtained from the patient, using MSP.

62. A method for treating a patient having colorectal and/or colon cancer, comprising:
I. assessing whether the MGMT gene is methylated, in a sample obtained from the patient, using MSP; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if methylation of the MGMT gene is detected in the sample.

63. The method of claim 12, in which the sample is a tumor biopsy sample.

64. A method of treating a patient having colorectal and/or colon cancer, comprising administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the MGMT protein is not detected in a sample obtained from the patient using a Western blot immunoassay, an immunohistochemical technique, or an enzymatic assay for MGMT protein.

65. A method for treating a patient having colorectal and/or colon cancer, comprising:
I. conducting a Western blot, an immunohistochemical assay or an enzymatic assay to detect the presence or absence of the MGMT protein in a sample obtained from the patient; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if MGMT protein is not detected in the sample.

66. The method of claim 5, in which the sample is a tumor biopsy sample.

67. A method of treating a patient having colorectal and/or colon cancer, comprising administering temozolomide to the patient according to one of the following three regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle
if the level of the MGMT protein or enzymatic activity detected in a sample obtained from the patient is Low or Moderate, compared to that of normal lymphocytes.

68. The method of claim 17, in which the sample is a tumor biopsy sample.

69. A method for treating a patient having colorectal and/or colon cancer, comprising:
I. assessing the level or enzymatic activity of the MGMT protein in a sample obtained from the patient and classifying the level or activity as Low, Moderate, or High as compared to that of normal lymphocytes; and
II. administering temozolomide to the patient according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patient's level or enzymatic activity of the MGMT protein is classified as Low or Moderate, compared to that of normal lymphocytes.

70. A method for treating a patient having colorectal and/or colon cancer, comprising:
I. assessing the level of methylation of the MGMT gene in a sample obtained from the patient and classifying the level as Low, Moderate, or High as compared to that of normal lymphocytes; and
II. administering temozolomide to the patient according to one of the regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle;
if the patients' level of methylation of the MGMT gene is classified as Moderate or High compared to that of normal lymphocytes.

71. A kit comprising reagents and instructions for conducting the method according to any of claims 1, 11, 21, 31, 41, 51, or 61.

72. A method for treating a patient having a glioma, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

73. A method for treating a patient having a melanoma, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

74. A method for treating a patient having a lung cancer, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

75. A method for treating a patient having a lymphoma, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

76. A method for treating a patient having head and neck cancer, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

77. A method for treating a patient having ovarian cancer, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

78. A method for treating a patient having colorectal and/or colon cancer, comprising:
I. assessing the enzymatic activity of the MGMT protein in a sample obtained from the patient and classifying the enzymatic activity as Low, Moderate, or High based on a comparison with cell lines known to express MGMT; and
II. administering temozolomide to the patient as follows:
a) if the patient's enzymatic activity of the MGMT protein is classified in step I as Low, according to one of the following two regimens:
i. 150-200 mg/m² per day for 5 days in a 28 day cycle; or
ii. 250 mg/m² per day for 5 days in a 28 day cycle in combination with a growth factor; or
b) if the patient's enzymatic activity of the MGMT protein is classified as Moderate, according to one of the following four regimens:
i. 100 mg/m² per day for 14 days in a 28 day cycle; or
ii. 300 mg/m² per day for 5 days in a 28 day cycle in combination with a growth factor; or
iii. 75 mg/m² day for 21 days in a 28 day cycle; or
iv. 75 mg/m² per 42 days in a 56 day cycle; or
c) if the patient's enzymatic activity of the MGMT protein is classified as High, according to one of the following regimens:
i. 100 mg/m² per day for 14 days in a 21 day cycle; or
ii. 150 mg/m² per day for 7 days in a 14 day cycle; or
iii. 100 mg/m² per day for 21 days in a 28 day cycle.

79. A kit comprising reagents and instructions for conducting the method according to any of claims 72-78.
